(19) United States
(12) Patent Application Publication
(10) Pub. No.: US 2024/0409960 A1
(43) Pub. Date: Dec. 12, 2024

(54) METHOD FOR CONTROLLING PROTEIN FUNCTION

(71) Applicant: The University of Tokyo, Tokyo (JP)

(72) Inventors: Moritoshi SATO, Tokyo (JP); Fuun KAWANO, Tokyo (JP)

(73) Assignee: The University of Tokyo, Tokyo (JP)

(21) Appl. No.: 18/702,120

(22) PCT Filed: Oct. 20, 2022
(86) PCT No.: PCT/JP2022/039087

§ 371 Date: Apr. 17, 2024

Related U.S. Application Data
(60) Provisional application No. 63/270,180, filed on Oct. 21, 2021.

(51) Int. Cl. C12N 15/90; C12N 9/22; C12N 15/11

(52) U.S. Cl. CPC C12N 15/902; C12N 9/22; C12N 15/11; C07K 2319/35; C12N 2310/20

(57) ABSTRACT

It is an object of the present invention to provide a method of reliably chemogenetically controlling the activity of a protein, and to provide a protein used in the present method. Specifically, the present invention relates to a plurality of protein fragments, which recover the activity of the protein when the plurality of protein fragments assemble, and in which each of the plurality of protein fragments is tagged with a degradation domain (DD) sequence. In addition, the present invention also relates to a method of regulating the activity of a protein in a cell, comprising: a step of introducing into a cell, a fusion of a DD sequence, and each fragment of a protein that has been split into fragments, such that the fragments recover the activity of the protein when they assemble; and a step of introducing a DD sequence-specific stabilizing factor into the cell.

### TECHNICAL FIELD

The present invention relates to a method of controlling protein function, and in particular, to a method of chemogenetically controlling protein function.

### BACKGROUND ART

Chemogenetic technique, which is utilized as a means for controlling protein function with chemical substances, is used as a method of performing genome editing utilizing site-specific DNA recombination technology, etc.

Since site-specific DNA recombination technology can induce recombination of a target DNA region with high accuracy, it is an effective tool for performing DNA editing in vitro and in vivo. Cre-loxP recombination system as a typical site-specific DNA recombination technology is a gene recombination system, which utilizes a site-specific recombination reaction caused by the specific action of a bacteriophage P1-derived tyrosine-type Cre recombinase on the DNA sequence called loxP. To date, a large number of inducible Cre recombinases, whose activity is induced under certain conditions, have been developed, and have been utilized for conditional genomic knock-in/knock-out operations. Among them, inducible Cre-loxP recombination systems utilizing Cre recombinases that become active in the presence of chemical substances, such as, for example, Cre-ER induced by tamoxifen, FKBP-FRB split-Cre induced by rapamycin, and Cre tagged with a degradation domain stabilized by field-1, have been developed and have already been utilized in genome editing in vivo.

Ligand-inducible DNA recombination systems, whose activity is induced by chemical substances, are tools that can be stably utilized to perform genome editing in vivo. Trimethoprim (TMP) has been utilized as a ligand for regulating the function of the Cre-loxP recombination system. TMP is an antibacterial ligand that stabilizes a degradation domain (DD) derived from E. coli dihydrofolate reductase (ecDHFR). The DD sequence has a property by which it induces the degradation of a protein, when it is tagged into the protein. However, in the presence of TMP, the degradation of the protein is inhibited. The destabilized Cre tagged with the DD sequence (in the absence of TMP, Cre is destabilized, and degraded in the proteasome) is called DD-Cre, and the DD-Cre has been developed for performing TMP-dependent inducible Cre-loxP recombination (Non Patent Literature 1 and Non Patent Literature 2).

TMP has low cytotoxicity and exhibits good cell membrane permeability and high tissue transportability, having no endogenous targets. Thus, a TMP-dependent Cre-loxP recombination system utilizing the DD sequence has been considered to be highly useful for application of a chemogenetic technique in vivo.

However, conventional DD-Cre has been reported to cause DNA recombination even in the absence of TMP (Non Patent Literature 1 and Non Patent Literature 2). In fact, since the conventional DD-Cre recombinase causes “leakage” of DNA recombination activity in the absence of TMP, it has been difficult to reliably control DNA recombination in vivo by the Cre-loxP recombination system utilizing DD-Cre.

### CITATION LIST

### Non Patent Literature

Non Patent Literature 1: Sando III et al., Nat. Methods 10:1085-1088, 2013.
Non Patent Literature 2: Pieraut et al., Neuron 84:107-122, 2014.

### SUMMARY OF INVENTION

### Technical Problem

Considering the aforementioned circumstances, it is an object of the present invention to provide a method of reliably chemogenetically controlling the activity of a protein, and a protein used in the present method.

### Solution to Problem

The present inventors have attempted to develop a Cre that does not cause DNA recombination in the absence of TMP, namely, a Cre that does not cause background activity of DNA recombination in the absence of TMP. Specifically, the present inventors have split a Cre recombinase into two fragments, i.e., one on the N-terminal side and the other on the C-terminal side, and have then fused a degradation domain (DD) sequence with each of the two fragments, so as to prepare a Cre recombinase consisting of the two fragments, and the present inventors have named it SPEED-Cre. Since the two Cre fragments constituting SPEED-Cre are each tagged with the DD sequence, they are destabilized in the absence of TMP and are degraded in the proteasome. On the other hand, in the presence of TMP, the two Cre fragments are stabilized, self-assemble without degradation, and exhibit recombinase activity. The present inventors have performed Cre-loxP recombination using SPEED-Cre in human cells and in living mouse bodies, and have found that only extremely low background DNA recombination activity is generated in the absence of TMP compared with a negative control, while DNA recombination is efficiently induced in the presence of TMP.

Furthermore, the present inventors have examined whether the properties of SPEED-Cre could also be confirmed in the case of using other proteins. Flp, VCre, and Dre, which are recombinases that perform site-specific DNA recombination, as with Cre, were each split into two fragments, and each fragment is then tagged with a DD repeated sequence. Then, the present inventors have examined whether these recombinases can induce TMP-dependent DNA recombination, and have found that, as with SPEED-Cre, these recombinases show only extremely low background activity in the absence of TMP, and induce efficient DNA recombination in the presence of TMP.

From these results, it is considered that the method for producing SPEED-Cre is highly likely to be applied to proteins other than the recombinases and is a useful new method for genome editing technology.

The present invention has been completed based on the aforementioned findings.

Specifically, the present invention includes the following (1) to (15).
(1) A plurality of protein fragments, which recover the activity of the protein when the plurality of protein fragments assemble, and in which each of the plurality of protein fragments is tagged with a degradation domain (DD) sequence.
(2) The plurality of protein fragments according to the above (1), wherein each fragment of the protein fragments has a binding region to one or multiple fragments other than the fragment.
(3) The plurality of protein fragments according to the above (2), wherein the binding region is an exogenous peptide.
(4) The plurality of protein fragments according to the above (3), wherein the exogenous peptide is a leucine zipper peptide or a dimer protein.
(5) The plurality of protein fragments according to the above (1), wherein the number of the plurality of protein fragments is 2.
(6) The plurality of protein fragments according to the above (1), wherein the protein is any one of a genome editing technology-related protein, a transcription factor-based protein, a reporter-based protein, a reprogramming-related protein, and a cell death-related protein.
(7) The plurality of protein fragments according to the above (1), wherein the DD sequence is any one of a DHFR-derived sequence, an FKBP-derived sequence, an FKBP12 mutant-derived sequence, and a UnaG-derived sequence.
(8) A nucleic acid encoding each fragment of the plurality of protein fragments according to the above (1).
(9) An expression vector having the nucleic acid according to the above (8).
(10) A genome editing kit, comprising the plurality of protein fragments according to the above (1) and/or the expression vector according to the above (9).
(11) The kit according to the above (10), wherein the plurality of protein fragments are site-specific recombinases, TALE proteins, or Cas9 nucleases.
(12) The kit according to the above (10), wherein the expression vector has a nucleic acid encoding each of the plurality of protein fragments that are site-specific recombinases, TALE proteins, or Cas9 nucleases.
(13) A method of regulating the activity of a protein in a cell, comprising:
   a step of introducing into a cell, a fusion of a degradation domain (DD) sequence, and each fragment of a protein that has been split into fragments, such that the fragments recover the activity of the protein when they assemble; and
   a step of introducing a DD sequence-specific stabilizing factor into the cell.
(14) The method according to the above (13), wherein the protein is any one of a genome editing technology-related protein, a transcription factor-based protein, a reporter-based protein, a reprogramming-related protein, and a cell death-related protein.
(15) The method according to the above (13), wherein the DD sequence is any one of a DHFR-derived sequence, an FKBP-derived sequence, an FKBP12 mutant-derived sequence, and a UnaG-derived sequence.

It is to be noted that the preposition “to” used in the present description indicates a numerical value range including the numerical values located left and right of the preposition.

### Advantageous Effects of Invention

According to the method of the present invention, it becomes possible to freely control the expression and function of proteins in a living body by using chemical substances.

### BRIEF DESCRIPTION OF DRAWINGS

FIG. 1 shows evaluation of the properties of DD-Cre. FIG. 1(a) shows an outline of the action mechanism of DD-Cre. In the absence of Trimethoprim (TMP), through the ubiquitin-proteasome pathway, a degradation domain (DD) derived from E. coli dihydrofolate reductase (ecDHFR) is tagged into iCre and induces degradation of the iCre. TMP stabilizes the structure of DD, suppresses the degradation of iCre, and causes recombination of the DNA sequence flanked by two loxP sites. NLS (nuclear localization signal) indicates a nuclear localization signal, CMV (cytomegalovirus promoter) indicates a cytomegalovirus promoter, and poly-A (3x) indicates a repeated sequence of a polyadenylation transcription “stop” signal. FIG. 1(b) shows the expression constructs of codon-optimized Cre (iCre) and DD-Cre. HA indicates the epitope sequence of an HA-tagged antibody. FIG. 1(c) shows the results of the measurement of the DNA recombination activity of DD-Cre and iCre in HEK293 cells in the absence of TMP (luciferase assay results). The term “reporter only” indicates the results obtained by transfecting a pcDNA 3.1 empty vector and a reporter gene into HKE293 cells as a negative control, and then measuring the activity. “n.s. (not significant)” indicates no significant difference. FIG. 1(d) shows the luciferase assay results of DNA recombination by DD-Cre in HEK293 cells after addition of TMP and DMSO at the concentrations shown in the figure. FIG. 1(e) shows the transfection ratio of DD-Cre, pcDNA 3.1 empty vector plasmid, and reporter plasmid used in the experiment of FIG. 1(f). FIG. 1(f) shows that TMP (10 nM or 500 nM) or DMSO was added to HEK293 cells, and that the DD-Cre expression plasmid was transfected in various amounts into the cells, followed by performing a luciferase assay. Data are shown as mean±s.d. (n=12). Dots are independent data points. Data represent the experimental results of three independent assays.

FIG. 2 shows evaluation of the properties of Cre tagged with a serial repeated sequence of DD sequence. FIG. 2(a) shows the expression constructs of DD-Cre tagged with a serial repeated sequence of the DD sequence. FIG. 2(b) shows the results of a luciferase assay of DNA recombination by DD-Cre (1x), DD-Cre 1.1 (3x), DD-Cre 1.2 (6x), DD-Cre 1.3 (9x) and DD-Cre 1.4 (12x) in HEK293 cells in the absence of TMP. *** p<0.001 is by Welch's two-tailed t-test. FIG. 2(c) shows the transfection ratio of DD-Cre 1.4, pcDNA 3.1 empty vector plasmid, and reporter plasmid used in the experiments of FIGS. 2(d) and (e). FIG. 2(d) shows that TMP (10 nM or 500 nM) or DMSO was added to HEK293 cells, and 24 hours after the addition, the DD-Cre 1.4 expression plasmid was transfected in various amounts into the cells, followed by performing a luciferase assay of DNA recombination. FIG. 2(e) shows that TMP (10 nM or 500 nM) or DMSO was added to HEK293 cells, and 48 hours after the addition, the DD-Cre 1.4 expression plasmid was transfected in various amounts into the cells, followed by performing a luciferase assay of DNA recombination. Data are shown as mean±s.d. (n=12). Dots are independent data points. Data represent the experimental results of three independent assays.

FIG. 3 shows production of a split Cre that self-assemble (spontaneously assemble). FIG. 3(a) shows the amino acid sequence of Cre recombinase. The split sites (Ser152 and Asp153) are underlined (SD). FIG. 3(b) shows the secondary structure of Cre recombinase. The N-terminal side (a.a. 2-152) and C-terminal side (a.a. 153-343) of the split Cre are shown, respectively. FIG. 3(c) shows the crystal structure of Cre recombinase. The split sites (Ser152 and Asp153) are indicated with arrows. FIG. 3(d) shows an outline of the action mechanism of self-assembling split Cre (sasCre). The N-terminus and C-terminus of the split Cre self-assemble, and the obtained sasCre recovers its enzyme activity and induces recombination of the DNA sequence flanked by the two loxP sites. FIG. 3(e) shows the expression constructs of sasCre and DD-sasCre. P2A indicates a 2A self-cleavage peptide for multiple gene expression. FIG. 3(f) shows the results of a luciferase assay of DNA recombination by sasCre and iCre in HEK293 cells in the absence of TMP. “n.s.” indicates no significant difference. FIG. 3(g) shows the results of a luciferase assay of DNA recombination by DD-Cre, DD-Cre 1.4 and DD-sasCre in HEK293 cells in the absence of TMP. * p<0.05 is by Welch's two-tailed t-test. Data are shown as mean+s.d. (n=12). Dots are independent data points. Data represent the experimental results of three independent assays.

FIG. 4 shows production of SPEED-Cre. FIG. 4(a) shows an outline of the action mechanism of SPEED-Cre. In the absence of TMP, through the ubiquitin-proteasome pathway, DD that is tagged into the N-terminal fragment and C-terminal fragment of SPEED-Cre strongly induces self-degradation of each fragment. TMP stabilizes the structure of DD, suppresses the degradation of SPEED-Cre, and induces recombination of the DNA sequence flanked by the two loxP sites without causing leakage of the recombination activity. FIG. 4(b) shows the expression constructs of SPEED-Cre and DD (12x)-sasCre. FIG. 4(c) shows DNA recombination by DD-Cre, SPEED-Cre and DD (12x)-sasCre in HEK293 cells in the absence of TMP, as measured by a luciferase assay. *** p<0.001 is by Welch's two-tailed t-test. “n.s.” indicates no significant difference. FIG. 4(d) shows that TMP (10 nM or 500 nM) or DMSO was added to HEK293 cells, and 24 hours or 48 hours after the addition, 5 ng of SPEED-Cre expression plasmid was transfected into the cells, followed by performing a luciferase assay. FIG. 4(e) shows the transfection ratio of SPEED-Cre, pcDNA 3.1 empty vector plasmid, and reporter plasmid used in the experiments of FIGS. 4(d), (f) and (g). FIG. 4(f) shows that TMP (10 nM or 500 nM) or DMSO was added to HEK293 cells, and that 24 hours after the addition, the SPEED-Cre expression plasmid was transfected in various amounts into the cells, followed by performing a luciferase assay. FIG. 4(g) shows that TMP (10 nM or 500 nM) or DMSO was added to HEK293 cells, and that 48 hours after the addition, the SPEED-Cre expression plasmid was transfected in various amounts into the cells, followed by performing a luciferase assay. Data are shown as mean+s.d. (n=12). Dots are independent data points. Data represent the experimental results of three independent assays.

FIG. 5 shows evaluation of the properties of SPEED-Cre in vivo (Part 1). FIG. 5(a) shows an outline of an in vivo experimental method using DD-Cre and SPEED-Cre. FIG. 5(b) shows the results obtained by imaging the in vivo bioluminescence of DD-Cre and SPEED-Cre after TMP treatment. Mice, into which a DD-Cre expression plasmid, a SPEED-Cre expression plasmid, a iCre expression plasmid or a pcDNA3.1 empty vector plasmid, 100 μL of TMP (167 μg/g body weight) or DMSO, and the reporter plasmid used in the experiments of FIG. 1 (Floxed-STOP Fluc) were injected, were used for the bioluminescence imaging of Fluc expression. FIG. 5(c) shows the results of bioluminescence measurements in vivo in the mice shown in FIG. 5(b) and FIG. 7. Data are shown as mean±s.d. (n=4). Dots are independent data points.

FIG. 6 shows studies regarding the applicability of a method for producing SPEED-Cre against various tyrosine-type recombinases. FIG. 6(a to c) shows an outline of the action mechanisms of SPEED-Flp (a), SPEED-VCre (b), and SPEED-Dre (c). The N-terminal fragment of split-Flp (a.a. 2-374) and the C-terminal fragment of split-Flp (a.a. 375-423), the N-terminal fragment of split-VCre (a.a. 2-277) and the C-terminal fragment (a.a. 278-380), and the N-terminal fragment of split Dre (a.a. 2-150) and the C-terminal fragment of split Dre (a.a. 151-342) are, respectively, self-assembled. FIG. 6(d to f) shows the expression constructs of DD-Flp and SPEED-Flp (d), the expression constructs of DD-VCre and SPEED-VCre (e), and the expression constructs of DD-Dre and SPEED-Dre (f). FIG. 6(g to i) shows that the expression plasmid of DD-Flp or SPEED-Flp (g), the expression plasmid of DD-VCre or SPEED-VCre (h), and the expression plasmid of DD-Dre or SPEED-Dre (i) were transfected in an amount of 5 ng each into HEK293 cells, and TMP (10 nM or 500 nM) or DMSO was then added to the HEK293 cells. DNA recombination activity was measured 24 hours after the transfection of the plasmids. FIG. 6(j to 1) shows that the expression plasmid of DD-Flp or SPEED-Flp (g), the expression plasmid of DD-VCre or SPEED-VCre (h), and the expression plasmid of DD-Dre or SPEED-Dre expression plasmid (i) were transfected in an amount of 5 ng each into HEK293 cells, and TMP (10 nM or 500 nM) or DMSO was then added to the HEK293 cells. DNA recombination activity was measured 48 hours after the transfection of the plasmids. Data are shown as mean±s.d. (n=12). Dots are independent data points. Data represent the experimental results of three independent assays.

FIG. 7 shows evaluation of the properties of SPEED-Cre in vivo (Part 2). Mice were injected with a DD-Cre expression plasmid, a SPEED-Cre expression plasmid, an iCre expression plasmid or a pcDNA3.1 empty vector plasmid, and were then administered with TMP (167 μg/g body weight) or DMSO. Thereafter, the reporter plasmid (Floxed-STOP Fluc) used in the experiment of FIG. 1 was intraperitoneally injected, and bioluminescence images were obtained. These images were used to create the graph shown in FIG. 5c. The images were selected from the results of two independent experiments.

FIG. 8 shows evaluation of the properties of SPEED-Flp, SPEED-VCre, or SPEED-Dre. FIG. 8(a to c) shows that DD-Flp, SPEED-Flp, Flp or a pcDNA3.1 empty vector plasmid (reporter only) (a), DD-VCre, SPEED-VCre, VCre or a pcDNA3.1 empty vector plasmid (reporter only) (b), and DD-Dre, SPEED-Dre, Dre or a pcDNA3.1 empty vector plasmid (reporter only) (c) were transfected in an amount of 5 ng each into HEK293 cells, and TMP (10 nM or 500 nM) or DMSO was then added to the HEK293 cells, and thereafter, a luciferase assay was performed 24 hours after the addition of TMP (10 nM or 500 nM) or DMSO. FIG. 8(d to f) shows that DD-Flp, SPEED-Flp, Flp or a pcDNA3.1 empty vector plasmid (reporter only) (d), DD-VCre, SPEED-VCre, Vcre or pcDNA3.1 empty vector (reporter only) (e), and DD-Dre, SPEED-Dre, Dre or a pcDNA3.1 empty vector plasmid (reporter only) (f) were transfected in an amount of 5 ng each into HEK293 cells, and TMP (10 nM or (10 nM or 500 nM) or DMSO was then added to the HEK293 cells, and thereafter, a luciferase assay was performed 48 hours after the addition of TMP (10 nM or 500 nM) or DMSO. Data are shown as mean+s.d. (n=12). Dots are independent data points. Data represent the experimental results of three independent assays.

FIG. 9 shows production of SPEED-Cas9. FIG. 9(a) shows an outline of the action mechanism of SPEED-Cas9. In the absence of TMP, through the ubiquitin-proteasome pathway, DD that is tagged into the N-terminal fragment (a.a. 2-713) and the C-terminal fragment (a.a. 714-1,368) of SPEED-Cas9 strongly induces self-degradation of each fragment. TMP stabilizes the structure of DD, suppresses the degradation of SPEED-Cas9, and reconstitutes RNA-induced nuclease activity. When Cas9 cleaves a luciferase reporter gene controlled by CMV at the site of an in-frame stop codon, the luciferase reporter gene is repaired by homologous recombination with a luciferase donor gene without having promoters, and bioluminescence activity is restored. FIG. 9(b) shows the expression constructs of full length (FL)-Cas9 and SPEED-Cas9. FIG. 9(c) shows the amino acid sequences of NZ and CZ hetero-leucine zippers and an outline of their mechanism of actions. NZ and CZ can assist reconstitution of non-self-assembling split proteins. FIG. 9(d) shows that TMP (500 nM) or DMSO was added to HEK293FT cells, and 48 hours after the addition, 12.5 ng of a SPEED-Cas9 expression plasmid was transfected into the HEK293FT cells, and thereafter, a luciferase-based HDR (Homology-directed repair) assay was performed. FIG. 9(e) shows that TMP (500 nM) or DMSO was added to HEK293FT cells, and 48 hours after the addition, 12.5 ng of a full-length Cas9 expression plasmid was transfected into the HEK293FT cells, and thereafter, a luciferase-based HDR (Homology-directed repair) assay was performed. Data are shown as mean±s.d. (n=16). Dots are independent data points. Data represent the experimental results of three independent assays.

FIG. 10 shows the nucleotide sequence of pcDNA3.1-Floxed-STOP Fluc (SEQ ID No: 1).

FIG. 11 shows the amino acid sequence of iCre (SEQ ID No: 2).

FIG. 12 shows the amino acid sequence of DD-Cre (SEQ ID No: 3).

FIG. 13 shows the amino acid sequence of DD-Cre 1.1 (SEQ ID No: 4).

FIG. 14 shows the amino acid sequence of DD-Cre 1.2 (SEQ ID No: 5).

FIG. 15 shows the amino acid sequence of DD-Cre 1.3 (SEQ ID No: 6).

FIG. 16 shows the amino acid sequence of DD-Cre 1.4 (SEQ ID No: 7).

FIG. 17 shows the amino acid sequence of sasCre (SEQ ID No: 8).

FIG. 18 shows the amino acid sequence of DD-sasCre (SEQ ID No: 9).

FIG. 19 shows the amino acid sequence of SPEED-Cre (SEQ ID No: 10).

FIG. 20 shows the amino acid sequence of DD (12x)-sasCre (SEQ ID No: 11).

FIG. 21 shows the nucleotide sequence of pcDNA3.1-Floxed-STOP mCherry (SEQ ID No: 12).

FIG. 22 shows the nucleotide sequence of pcDNA3.1-Floxed (FRT)-STOP Fluc (SEQ ID No: 13).

FIG. 23 shows the amino acid sequence of Flp (SEQ ID No: 14).

FIG. 24 shows the amino acid sequence of DD-Flp (SEQ ID No: 15).

FIG. 25 shows the amino acid sequence of SPEED-Flp (SEQ ID No: 16).

FIG. 26 shows the nucleotide sequence of pcDNA3.1-Floxed (Vlox)-STOP Fluc (SEQ ID No: 17).

FIG. 27 shows the amino acid sequence of VCre (SEQ ID No: 18).

FIG. 28 shows the amino acid sequence of DD-VCre (SEQ ID No: 19).

FIG. 29 shows the amino acid sequence of SPEED-VCre (SEQ ID No: 20).

FIG. 30 shows the nucleotide sequence of pcDNA3.1-Floxed (rox)-STOP Fluc (SEQ ID No: 21).

FIG. 31 shows the amino acid sequence of Dre (SEQ ID No: 22).

FIG. 32 shows the amino acid sequence of DD-Dre (SEQ ID No: 23).

FIG. 33 shows the amino acid sequence of SPEED-Dre (SEQ ID No: 24).

FIG. 34 shows the nucleotide sequence of pcDNA3.1-V5-HisA-StopFluc (SEQ ID No: 25).

FIG. 35 shows the nucleotide sequence of pCold-I-inverted-StopFluc (SEQ ID No: 26).

FIG. 36 shows the structure of a gRNA expression vector and the nucleotide sequence of gRNA (SEQ ID No: 29). The gRNA targeting StopFluc was produced by performing annealed oligo cloning, utilizing the BbsI site of a pU6-gRNA expression vector (Addgene: 47108).

FIG. 37 shows the amino acid sequence of full-length spCas9 (SEQ ID No: 27).

FIG. 38 shows the amino acid sequence of SPEED-Cas9 (SEQ ID No: 28).

### DESCRIPTION OF EMBODIMENTS

Hereafter, the embodiments for carrying out the present invention will be described.

A first embodiment relates to a plurality of protein fragments, which recover the activity of the protein when the plurality of protein fragments assemble, and in which each of the plurality of protein fragments is tagged with a degradation domain (DD) sequence (hereinafter also referred to as “the protein fragment according to the present embodiment”).

The “protein” of the present embodiment (i.e., the original protein before fragmentation into multiple fragments) may be any protein, as long as its activity or function can be confirmed. The “protein” of the present embodiment is not particularly limited, and examples of the protein of the present embodiment may include: genome editing technology-related proteins, such as site-specific recombinases including tyrosine-type recombinases (for example, Cre, VCre, SCre, Dre, Flp, Vika, KD, B2, B3, λ-Int, HK022, HP1, etc.) and serine-type recombinases (for example, φC31, Bxb1, R4, etc.), TALE proteins such as ZEN and TALEN, and CRISPR-Cas-based nucleases (for example, Cas9 nuclease, etc.); transcription factor proteins, such as transcription regulators (for example, VP16, p65, HSF1, etc.) and transcription repressors (for example, KRAB, etc.); reporter proteins, such as fluorescent proteins (for example, GFP, Cherry, etc.), luciferases (for example, Fluc, Nanoluc, etc.), and antibodies, antibody-like proteins and antibody-like peptides (for example, IgG, scFv, nanobody, monobody, etc.); reprogramming-related proteins, such as proteins involved in cell reprogramming (for example, Nanog, Oct3/4, Sox2, Klf4, c-Myc, etc.) and proteins involved in direct conversion (for example Myod, C/EBPα, etc.); and cell death-related proteins, such as apoptosis-related proteins (for example, Caspase 3, Caspase 9, etc.) and necroptosis-related proteins (for example, MLKL, etc.).

In a cell, a protein having DD has a property of being degraded by proteasome, but in the presence of a DD-stabilizing substance that is specific for each DD, the degradation of the protein is suppressed. Accordingly, the protein fragment of the present embodiment, which is tagged with a DD sequence, is degraded by proteasome in the absence of a DD-stabilizing substance, whereas in the presence of the DD-stabilizing substance, the DD binds to the stabilizing substance, so that the protein fragment can be stably present in the cell without being degraded.

The DD used in the present embodiment is not particularly limited, and a sequence known in the present technical field can be used. Examples of the combination of DD and a stabilizing substance thereof may include: a combination of a dihydrofolate reductase (DHFR)-derived DD sequence and Trimethoprim (TMP) (Sando III et al., Nat. Methods 10:1085-1088, 2013); a combination of FKBP (FK506-binding protein)-derived DD and Shield-1 (Banaszynski et al., Cell 126:995-1004, 2006); a combination of an FKBP12 mutant-derived DD sequence and Shield-1 or Shield-2 (Grimley et al., Bioorg Med Chem Lett. 18:759-761, 2008): a combination of an FKBP12 mutant-derived DD sequence and Rapamycin (Inoue et al., Nature Methods 2:415-518, 2005), a combination of a UnaG-derived DD sequence and bilirubin (Navarro et al., ACS Chemical Biology 11:2101-2104, 2016); and a combination of a mouse DHFR-derived DD sequence and methotrexate (Rajagopalan et al., Proc Natl Acad Sci USA. 99:13481-13486, 2002)

In addition, specific examples of the DD sequence are shown below. Wild-type ecDHFR-derived sequence (SEQ ID No: 30):

MISLIAALAVDRVIGMENAMPWNLPADLAWFKRNTLNKPVIMGRHTWESI
GRPLPGRKNIILSSQPGTDDRVTWVKSVDEAIAACGDVPEIMVIGGGRVY
EQFLPKAQKLYLTHIDAEVEGDTHFPDYEPDDWESVFSEFHDADAQNSHS
YCFEILERR

Mutant ecDHFR-derived sequence (SEQ ID No: 31: used in the Examples):

MISLIAALAVDYVIGMENAMPWNLPADLAWFKRNTLNKPVIMGRHTWESI
GRPLPGRKNIILSSQPSTDDRVTWVKSVDEAIAACGDVPEIMVIGGGRVI
EQFLPKAQKLYLTHIDAEVEGDTHFPDYEPDDWESVFSEFHDADAQNSHS
YCFEILERR

Human FKBP12-derived sequence (SEQ ID No: 32):

MGVQVETISPGDGRTFPKRGQTCVVHYTGMLEDGKKFDSSRDRNKPFKFM
LGKQEVIRGWEEGVAQMSVGQRAKLTISPDYAYGATGHPGIIPPHATLVF
DVELLKLE

Human FKBP12 mutant-derived sequence (F37V) (SEQ ID No: 33):

MGVQVETISPGDGRTFPKRGQTCVVHYTGMLEDGKKVDSSRDRNKPFKFM
LGKQEVIRGWEEGVAQMSVGQRAKLTISPDYAYGATGHPGIIPPHATLVF
DVELLKLE

UnaG-derived sequence (SEQ ID No: 34):

MVEKFVGTWKIADSHNFGEYLKAIGAPKELSDGGDATTPTLYISQKDGDK
MTVKIENGPPTFLDTQVKFKLGEEFDEFPSDRRKGVKSVVNLVGEKLVYV
QKWDGKETTYVREIKDGKLVVTLTMGDVVAVRSYRRATE

Mouse DHFR-derived DD sequence (SEQ ID No: 35):

MVRPLNCIVAVSQNMGIGKNGDLPWPPLRNEFKYFQRMTTTSSVEGKQNL
VIMGRKTWFSIPEKNRPLKDRINIVLSRELKEPPRGAHFLAKSLDDALRL
IEQPELASKVDMVWIVGGSSVYQEAMNQPGHLRLFVTRIMQEFESDTFFP
EIDLGKYKLLPEYPGVLSEVQEEKGIKYKFEVYEKKD

It is to be noted that the number of DD sequences to be tagged into protein fragments is not particularly limited, as long as it is 1 or more.

Moreover, a plurality of fragments of the protein according to the present embodiment exhibit the function of the original protein by assembling, and the assembly may be spontaneous assembly (self-assembly) or induced assembly. Furthermore, the “plurality” of the plurality of proteins means preferably, for example, 2 to 5, and more preferably 2 or 3.

To illustrate the case of spontaneous assembly of protein fragments, taking Cre recombinase as an example, when a protein is split into two protein fragments, for example, at a site between Ser152 and Asp153 in the C-terminal globe, these two fragments spontaneously assemble, and recombinase activity is recovered. Similarly, when Flp is split between the 374th and 375th amino acids, VCre is split between the 277th and 278th amino acids, and Dre is split between the 150th and 342nd amino acids, respectively, individual fragments spontaneously assemble, and recombinase activity is recovered. It is to be noted that the split position is not particularly limited, and in the case of Cre recombinase, examples of the split position may include between Leu26 and Met27, between Pro105 and Arg106, between Ala275 and Lys276, and between Gly280 and Gln281.

As mentioned above, in the present embodiment, with regard to the assembly of protein fragments, the protein fragments originally have a region in which they bind to each other, and the protein fragments may spontaneously assemble (self-assemble) through the binding region, or may inducibly assemble. In the case of inducible assembly, for example, exogenous peptides that bind to each other are allowed to fuse with individual protein fragments, so that the exogenous peptides become a binding region, and assembly may be induced via the exogenous peptides. These exogenous peptides may be peptides (or proteins) that form a dimer, or may also be peptides (or proteins) that form oligomers greater than the dimer. The exogenous peptide used herein is not particularly limited, and examples thereof may include: heterodimer-forming leucine zipper peptide (Leucine-Zipper) pairs, such as split-sfGFP (Pedelacq et al., Int. J. Mol. Sci. 2019 20:3479 doi: 10.3390/ijms20143479), FosLZ-JunLZ (Waraho et al., Proc Natl Acad Sci USA. 2009 106:3692-3697 doi: 10.1073/pnas.0704048106), and Lzk-LzE; dimer protein pairs, such as Fv-Clasp (Arimori et al., Structure 2017, 25:1611-1622. doi.org10.1016j.str.2017.08.011), and DocS-Coh2 (Yu et al., Sci Adv. 2020, 10: 6 (28): eabb1777. doi: 10.1126/sciadv); split-intein (Wang et al., Sci Rep. 2012, 2:497.doi: 10.1038/srep00497), etc.; homotrimer-forming GCN4 Leucine-Zipper (Oshaben et al., Biochemistry 2012, 27:9581-9591. doi: 10.1021/bi301132k), etc.; and other oligomer-forming AzamiGreen (Karasawa et al., J Biol Chem. 2003, 278:34167-34171. doi: 10.1074/jbc.M304063200) system, DsRed-Express 2 (Strack et al., Nat Methods 2008, 5:955-957. doi: 10.1038/nmeth.1264) system, CAD (Furuya et al., ACS Synth Biol. 2017, 16; 1086-1095. doi: 10.1021/acssynbio.7b00022) system, and the like. Therefore, “the protein fragment according to the present embodiment” may be tagged with the above-described exogenous peptides (or exogenous proteins).

Further, depending on the purpose of use of the present protein fragment, the protein fragment may also be tagged with a migration signal peptide, etc. for transferring the protein fragment to the nucleus or intracellular organelles, or with other peptides or proteins, such as antibody fragments or tag peptides.

A second embodiment relates to a nucleic acid (DNA or RNA) that encodes the protein fragment according to the present embodiment (hereinafter also referred to as “the nucleic acid according to the present embodiment”).

The nucleic acid according to the present embodiment can be prepared according to a method known in the present technical field or a method obtained by appropriately modifying such a known method. The nucleic acid according to the present embodiment is incorporated into a suitable expression vector, suitable host cells are then transformed with the expression vector, the host cells are then cultured in a suitable medium, and the expressed protein is then collected and purified, so that a protein fragment of interest can be obtained.

Examples of the host cells used for protein expression may include bacterial cells (for example, Escherichia coli B strain, E. coli K12 strain, Corynebacterium ammoniagenes, C. glutamicum, Serratia liquefaciens, Streptomyces lividans, Pseudomonas putida, etc.), molds (for example, Penicillium camembertii, Acremonium chrysogenum, etc.), animal cells, plant cells, and baculovirus/insect cells or yeast cells (for example, Saccharomyces cerevisiae and Pichia pastoris, etc.).

Besides, the host cells expressing a protein may be used for the purpose of controlling the function of the protein in the host cells, other than for the purpose of preparation of the protein.

As expression vectors for expressing proteins, vectors suitable for various types of host cells can be used. Examples of the expression vector that can be used herein may include: pBR322, pBR325, pUC118, pET, etc. (Escherichia coli hosts): pEGFP-C, pEGFP-N, pcDNA3.1, etc. (animal cell hosts): pVL1392, pVL1393, etc. (insect cell hosts, baculovirus vectors); and pG-1, Yep13, pPICZ, etc. (yeast cell hosts). These expression vectors have a replication origin, a selective marker, and a promoter, which are suitable for each vector. These expression vectors may also have an enhancer, a transcription termination sequence (terminator), a ribosome binding site, a polyadenylation signal, etc., as necessary. Further, in order to facilitate purification of the expressed polypeptide, a nucleotide sequence for fusing a FLAG tag, a His tag, an HA tag, a GST tag, etc. with the polypeptide and then allowing the polypeptide to express may be inserted into such an expression vector.

Such an expression vector can be produced by a method known to a person skilled in the art, using a commercially available kit, as appropriate.

When the expressed protein is extracted from cultured cell masses or cultured cells, the cell masses or the cultured cells are collected by a known method after completion of the culture, and the collected cell masses or cells are then suspended in a suitable buffer. Thereafter, the suspension is subjected to ultrasonic wave, lysozyme and/or freezing-thawing, etc., so that the cell masses or cells are disintegrated. Thereafter, the resultant is subjected to centrifugation or filtration to obtain a soluble extract. In particular, when cultured cells are used as hosts, a method of obtaining a protein expressed in a culture supernatant by recovering the supernatant is desirable. An appropriate combination of known separation and/or purification methods is applied to the obtained extract or culture supernatant, so as to obtain a protein of interest. Examples of the known separation and/or purification methods that can be used herein may include: methods of utilizing solubility, such as salting-out or a solvent precipitation method: methods of mainly utilizing a difference in molecular weights, such as a dialysis method, an ultrafiltration method, a gel filtration method, or SDS-PAGE; methods of utilizing a difference in electric charges, such as ion exchange chromatography: methods of utilizing specific affinity, such as affinity chromatography (for example, methods, in which when a polypeptide is expressed together with a GST tag, a glutathione-bound carrier resin is used, when a polypeptide is expressed together with a His tag, a Ni-NTA resin or a Co-based resin is used, when a polypeptide is expressed together with a HA tag, an anti-HA antibody resin is used, and when a polypeptide is expressed together with a FLAG tag, an anti-FLAG antibody-bound resin or the like is used); methods of utilizing a difference in hydrophobicity, such as reverse phase high performance liquid chromatography; and methods of utilizing a difference in isoelectric points, such as an isoelectric focusing method.

A third embodiment relates to a genome editing kit, comprising the protein fragment according to the present embodiment and/or a vector that expresses the present protein fragment (hereinafter also referred to as “the kit according to the present embodiment”). The “genome editing” in the present embodiment means not only changing the structure of genomic DNA sequences (e.g., recombination, deletion, insertion, etc.) but also controlling the transcriptional activity of protein-coding DNA, for example, activating or suppressing the functions of a promoter, an enhancer, or a silencer, and also, performing epigenetic control, etc.

The kit according to the present embodiment may comprise, in addition to the protein fragment according to the present embodiment, chemical substances that stabilize the DD sequence (for example, TMP, Shield-1, Shield-2, Rapamycin, methotrexate, bilirubin, etc.). Although it is not particularly limited, when the kit according to the present embodiment is a kit for genome editing by site-specific DNA recombination, “the protein fragments according to the present embodiment” may be, for example, recombinase fragments that recover recombinase activity when they assemble, and they may be specifically the fragments of, for example, Cre, VCre, Dre, and Flp. Moreover, in addition to recombinase, the kit according to the present embodiment may also comprise protein fragments such as a TALE protein of TALEN and a Cas9 nuclease of CRISPR-Cas9, which function as site-specific nucleases, or a vector expressing such protein fragments.

Furthermore, the kit according to the present embodiment may also comprise other reagents (e.g. a buffer, restriction enzymes, a vector for protein expression, etc.), experimental equipment, and the like, which are necessary for genome editing.

A fourth embodiment relates to a method of regulating the activity of a protein in a cell (or a method of controlling protein function), comprising:
a step of introducing into a cell, a fusion of a degradation domain (DD) sequence, and each fragment of a protein that has been split into fragments, such that the fragments recover the activity of the protein when they assemble; and
a step of introducing a DD sequence-specific stabilizing factor into the cell.

When the protein fragments according to the present embodiment is introduced into a cell, the plurality of protein fragments are degraded by proteasome in the absence of a DD sequence-specific stabilizing substance. As a result, the original protein (i.e. a protein in which the plurality of protein fragments assemble) does not exhibit its activity, and it does not function in the cell. On the other hand, in the presence of a DD-specific stabilizing factor (for example, TMP in the case of using the DHFR-derived DD sequence of E. coli), the plurality of protein fragments assemble without being degraded, and the original protein can exhibit its activity in the cell and can function.

Therefore, the fourth embodiment relates to a method of regulating a desired protein in a cell by suppressing the activity of the desired protein in the absence of a stabilizing factor and recovering the activity of the protein in the presence of the stabilizing factor.

It is to be noted that the term “cell” includes both in vitro cells and in vivo cells. In addition, the method of introducing the protein fragment according to the present embodiment into a cell can be selected, as appropriate, by those skilled in the art. For example, a vector expressing each protein fragment may be introduced into a cell, and each protein fragment may be expressed in the cell. Otherwise, each protein fragment may be directly introduced into a cell. In a case where the present protein fragment is transferred into intracellular nucleus or intracellular organelles, a migration signal peptide (e.g. a nuclear localization signal peptide, a mitochondrial localization signal peptide, etc.) may be allowed to fuse with each protein fragment, and the protein fragment may be then introduced into the cell in such a state.

As described above, the method of introducing the protein fragment according to the present embodiment into a cell typically involves a method of introducing an expression vector having a protein fragment into a cell and then allowing the protein fragment to express in the cell. In some cases, however, the protein fragment may be directly introduced into the cell, using a cell membrane-permeable peptide, etc.

When the present description is translated into English and the English description includes singular terms with the articles “a,” “an,” and “the,” these terms include not only single items but also multiple items, unless otherwise clearly specified from the context that it is not the case.

Hereinafter, the present invention will be further described in the following examples. However, these examples are only illustrative examples of the embodiments of the present invention, and thus, are not intended to limit the scope of the present invention.

### EXAMPLES

### I. Analysis of SPEED-Cre, SPEED-Flp, SPEED-VCre, and SPEED-Dre

### 1. Methods

### 1-1. Reagents, Etc.

The oligonucleotides used for construction of plasmids were synthesized by Eurofines Genomics, Integrated DNA, GenScript, or ThermoFisher Scientific. The mammalian expression vector, pcDNA3.1 (+), was purchased from ThermoFisher Scientific. All plasmids were constructed based on individual methods, namely, an overlapping PCR extension method, the cleavage of a PCR product with restriction enzymes, and ligation of double-stranded DNA with T4 DNA ligase. The PCR reaction was performed using DNA polymerase of KOD One PCR Master Mix (TOYOBO) according to the manufacturer's instructions. All restriction enzymes were obtained from New England bioLabs, and were used according to the manufacturer's instructions. As T4 ligase, Ligation high Ver.2 (TOYOBO) was used. All the constructed plasmids were checked by DNA sequencing analysis service

### (Eurofines Genomics).

### 1-2. Construction of Plasmids

For constructing destabilized Cre, sequences encoding a degradation domain (DD) derived from E. coli dihydrofolate reductase (ecDHFR) tagged with an HA-tag antibody epitope sequence (HA), a nuclear localization signal (NLS), a P2A self-cleaving 2A peptide, or Cre recombinase encoded by codons optimized for mammalian expression were synthesized by Eurofines Genomics, Integrated DNA Technology, GenScript, or ThermoFisher Scientific. The sequence encoding Cre recombinase was inserted into the NheI-XbaI sites in a pcDNA3.1 (+) vector having a CMV promoter.

For constructing a luciferase reporter for detecting Cre-loxP recombinase activity, the polyadenylation signal (poly(A)) repeated sequence (“stop”)) flanked by two loxP sites (ataacttcgtatagcatacattatacgaagttat: SEQ ID No: 36) in the same orientation was amplified from Addgene plasmid (ID: 22797). Sequence encoding firefly luciferase (Fluc) was amplified from pGL4.31 vector (Promega). The loxP stop-loxP-Fluc construct was inserted into HindIII and XhoI sites in pcDNA3.1 (+) vector containing the CMV promoter.

For constructing a fluorescent protein for detecting Cre-loxP recombinase activity, sequence encoding mCherry was inserted into EcoRI and XhoI sites in the 548 pcDNA3.1 (+) of the luciferase reporter (of loxP-stop-loxP-Fluc).

For constructing destabilized Flp, VCre and Dre, the sequences encoding the DD, NLS, P2A, and/or Flp, VCre and Dre with codons optimized for mammalian expression were synthesized by Eurofines Genomics. The sequences encoding Flp, VCre and Dre were inserted into HindIII and XbaI sites in the pcDNA3.1 (+) vectors of iCre, DD-Cre, and Speed Cre.

For constructing the luciferase reporters detecting for the activities of Flp-FRT recombinase, VCre-Vlox recombinase and Dre-rox recombinase, the FRT site (gaagttcctatactttctagagaataggaacttc: SEQ ID No: 37), the Vlox site (tcaatttccgagaatgacagttctcagaaattga: SEQ ID No: 38), the rox site (taactttaaataattggcattatttaaagtta: SEQ ID No: 39), were inserted into HindIII and KpnI sites and ClaI and EcoRI sites in the pcDNA3.1 (+) vector of the luciferase reporter (of loxP-stop-loxP-Fluc).

### 1-3. Cell Culture

HEK293T cells (American Type Culture Collection: ATCC) were cultured at 37° C. under 5% CO₂ in Dulbecco's Modified Eagle Medium (DMEM; Thermo Fisher Scientific) supplemented with 10% fetal bovine serum (FBS; Thermo Fisher Scientific), 100 units/ml of penicillin and 100 μg/ml of streptomycin (Thermo Fisher Scientific) and 2 mM GlutaMAX (Thermo Fisher Scientific).

### 1-4. Site-Specific Recombination Experiments In Vitro

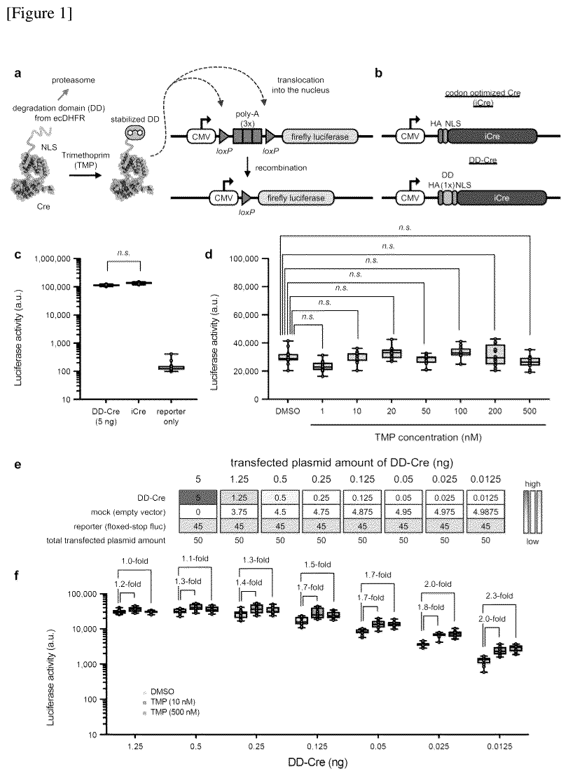

For luciferase assay, HEK 293 cells were plated at 25,000 cells per well on a 96-well flat white microplate (Thermo Fisher Scientific), and were then cultured for 24 hours at 37° C. in 5% CO₂. The cells were transfected with cDNAs encoding destabilized Cre along with the luciferase reporter (loxP-stop-loxP Fluc), using X-tremeGENE9 reagent (Merck Millipore) at 37° C. The total amount of DNA was 50 ng per well. The transfection ratio of destabilized Cre, pcDNA3.1 empty vector, and the luciferase reporter is as shown in FIGS. 1e, 2c, and 4e. Twenty-four hours after the transfection, TMP or DMSO was added into the cells, and the cells were further cultured for 24 hours or 48 hours. Just before the luciferase assay, the medium was replaced with Hanks' balanced salt solution (HBSS) (Thermo Fisher Scientific) containing 0.2 mM D-luciferin potassium salt. Bioluminescence measurement was performed for 1 second per well at room temperature using a Centro XS³ LB 960 plate-reading luminometer (Berthold Technologies).

For fluorescence imaging, HEK293 cells were plated on 35 mm glass bottom dishes (glass diameter: 14 mm) (Matsunami), and were then cultured for 24 hours at 37° C. in 5% CO². The cells were transfected with cDNAs encoding destabilized Cre along with the fluorescent protein reporter (loxP-stop-loxP mCherry) at 1:9 ratio, using X-tremeGENE9 reagent (Merck Millipore) at 37° C. The total amount of DNA was 100 ng per well. Twenty-four hours after the transfection, TMP or DMSO was added into the cells, and the cells were then cultured for further 24 hours. Just before the fluorescence imaging, the medium was replaced with HBSS containing 8.115 μM Hoechst 33342 nucleic acid stain (Invitrogen), and were then incubated for 30 minutes at room temperature. Then, in order to remove the staining solution, the cells were washed 3 time by HBSS. Fluorescence imaging was conducted with a LSM 710 confocal laser scanning microscope equipped with a 20× objective (Carl Zeiss). Fluorescence images of Hoechs and mCherry were taken using a Nm Diode laser (405 nm) and a HeNe laser (543 nm).

### 1-5. Site-Specific Recombination Experiments In Vivo

Experiments using mice were performed in accordance with the Guidelines for Care and Use of Laboratory Animals as stated by the University of Tokyo. In all the experiments, four week-old female Slc: ICR mice (Sankyo Labo Service Corporation, Inc.) were used. The mice were intraperitoneally injected with cDNAs encoding destabilized Cre along with the luciferase reporter (loxP-stop-loxP Fluc) at 1:9 ratio, using TransIT-EE Hydrodynamic Delivery Solution (Mirus Bio LLC, Madison). The total amount of the DNA injected into a mouse was 10 μg per mouse. The volume of a delivery solution was set at 0.1 mL per mouse weight (g). After the hydrodynamic (HTV) injection of DNA, the mice were bred for 3 hours, and thereafter, TMP (167 μg/g body weight) or DMSO was intraperitoneally (I.P.) injected into the mice. The total volume of the TMP solution injected into a mouse was 100 μL per mouse weight (g). The intraperitoneally injected mice were bred for further 21 hours. The bioluminescence imaging of the mice was performed 24 hours after the HTV injection of DNA. Prior to the bioluminescence imaging, 100 mM D-luciferin was intraperitoneally injected into the mice. The total volume of the injected D-luciferin solution was 10 μL per mouse weight (g). Three minutes after the D-luciferin injection, the bioluminescence images of the mice were obtained using the Lumazone bioluminescence imager (Nippon Roper) equipped with the Evolve EMCCD camera (Photometrics) for 1 minutes. During the intraperitoneal D-luciferin injection and the bioluminescence imaging, the mice were anaesthetized with isoflurane (Abb Vie) to keep them motionless. The obtained data were analyzed with the SlideBook 4.2 software (Intelligent Innovations Inc.).

### 1-6. Reproducibility and Statistics Analysis

No power studies were performed in cell culture experiments. None of samples were excluded from analysis. A method of randomization was performed in the cell culture experiments. No blinding was used in the cell culture experiments. In FIGS. 1c, 1d, 2b, 3f, 3g and 4c, variances estimated by F-tests were unequal. Statistical significance (P value) was determined using a two-tailed Welch's t-test. P values of less than 0.05 were considered significant.

### 2. Results

### 2-1. Evaluation of Properties of DD-Cre

In order to improve the problem of DD-Cre, first of all, a DD-Cre expression plasmid, and a full-length Cre recombinase (iCre) with codon optimized for mammalian expression (Shimshek et al., Genesis 32:19-26, 2002) expression plasmid, were constructed, and the recombination activity of DD-Cre was then confirmed in vitro (FIGS. 1a and b).

HEK 293 cells were transiently transfected with the DD-Cre expression plasmid along with a luciferase reporter gene. In the luciferase reporter gene used herein, two loxP sites were located in the upstream region of a firefly luciferase gene controlled by the CMV promoter, and a polyadenylation transcriptional “stop” signal (poly-A) repeated sequence was inserted between these loxP sites. As a result of reporter assay, the DNA recombination activity of DD-Cre was detected at almost the same level of the recombination activity of iCre even in the absence of TMP (FIG. 1c). Moreover, it was also found that TMP treatment at any concentration does not induce the DNA recombination by DD-Cre in a case where the cells were transfected with 5 ng of the DD-Cre expression plasmid (FIG. 1d).

Regarding the aforementioned results, the present inventors hypothesized that the occurrence of the leakage of DNA recombination activity by DD-Cre in the absence of TMP and extremely low DNA recombination activity induced in the presence of TMP would be caused by the overexpression of DD-Cre in the HEK 293 cells. Thus, the cells were transfected with a pcDNA3.1 empty vector plasmid, instead of with the DD-Cre expression plasmid, so that the expression level of DD-Cre was attempted to be decreased (FIG. 1e). However, unexpectedly, even when the transfection amount of DD-Cre was reduced by one 400th, the recombination activity by DD-Cre remained at almost the same level as that before the reduction of the transfection amount (FIG. 1f).

These results indicate that DD-Cre does not effectively function at least in vitro.

### 2-2. Development of DD Repeated Sequence-Tagged Cre

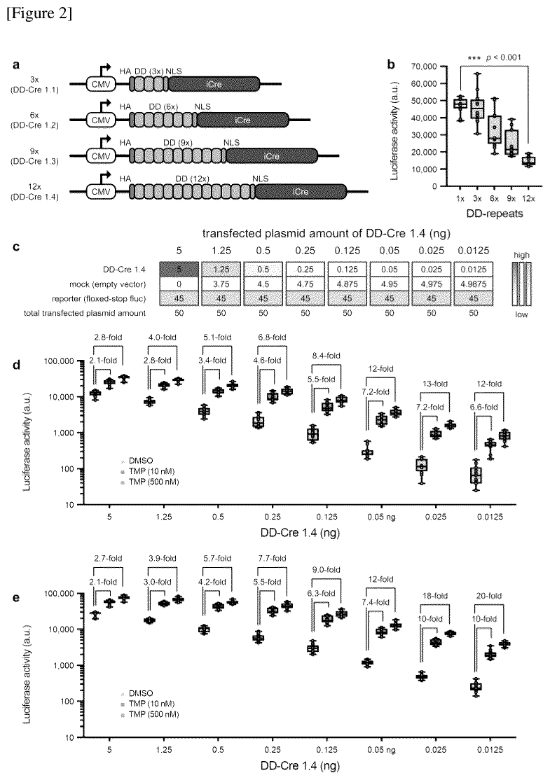

Next, whether or not the degradation efficiency of Cre could be improved by increasing the number of DD sequences to be tagged was examined (FIG. 2a). In the absence of TMP, the background DNA recombination activity of Cre tagged with 12 DD sequences (DD-Cre 1.4) was decreased to one fourth of the activity of DD-Cre (tagged with one DD) (FIG. 2b). These results show that the background activity of Cre is decreased by tagging it with multiple DD sequences.

On the other hand, the TMP-dependent DNA recombination activity of DD-Cre 1.4 still remained low in a case where the cells were transfected with 5 ng of the Cre expression plasmid (FIGS. 2c, d and e). Thus, the cells were transfected with a pcDNA3.1 empty vector plasmid, instead of with the DD-Cre expression plasmid, so that the expression level of DD-Cre 1.4 was decreased, and thereafter, the DNA recombination activity of DD-Cre 1.4 was measured (FIG. 2c). It was found that the leakage of the DNA recombination activity by DD-Cre 1.4 in the absence of TMP was decreased, as the amount of the DD-Cre 1.4 expression plasmid was decreased (FIGS. 2c, d and e). Moreover, when the cells were transfected with 0.05, 0.025 or 0.0125 ng of the DD-Cre 1.4 expression plasmid, the TMP-dependent DNA recombination activity was detected with a high induction efficiency of more than 10-fold (FIGS. 2d and d). The induction efficiency of the DNA recombination activity of DD-Cre 1.4 was more improved by TMP treatment for 48 hours (FIGS. 2c and e).

These results indicate that the leakage of DNA recombination activity by DD-Cre in the absence of TMP is decreased by tagging Cre with multiple DD sequences, and that the induction efficiency of TMP-dependent DNA recombination activity is improved.

### 2-3. Production of Speed-Cre

Next, the reason why DD-Cre 1.4 is not completely degraded despite the tagging with multiple DD sequences was examined. A previous study has shown that protein degradation of a poly-ubiquitinated protein through the ubiquitin-proteasome pathway requires sufficient time dependent on the molecular size of the protein: for example, it takes at least 6 hours for GFP consisting of 238 amino acid residues (29 kDa) to be completely degraded in HEK293 cells (Shabek et al., Mol. Cell 48:87-97, 2012). Thus, the present inventors hypothesized that Cre recombinase (38 kDa) larger than GFP requires at least several hours (6 hours or more) to be completely degraded. Cre can recombine the loxP sites at an efficiency of approximately 50% within 1 hour. Accordingly, it is quite possible that a certain degree of DNA recombination is performed before Cre is completely degraded, and that the leakage of recombination activity is detected. Therefore, it was considered that the tagging of Cre with multiple DD sequences contributes to increase the frequency of degradation, but that, on the contrary, it may cause a decrease in the efficiency of degradation.

In order to improve the degradation efficiency of Cre tagged with DD sequences, the present inventors examined the “split protein approach” for decreasing the protein size. In general, truncated proteins are structurally unstable and are easily degraded. In fact, a previous study has shown that truncated proteins were degraded at a faster rate compared with full-length proteins (Saeki et al., J. Biochem. 161:113-124, 2017). In addition, in the case of Cre recombinase, it is considered that the tetramer formation (Cre functions as a tetrameric recombinase) is inhibited by the truncating thereof, and that binding to the loxP site becomes impossible.

Therefore, it can be expected that the binding of split Cre to the loxP site is inhibited and the leakage of DNA recombination activity is suppressed, even if the split fragment is not completely degraded.

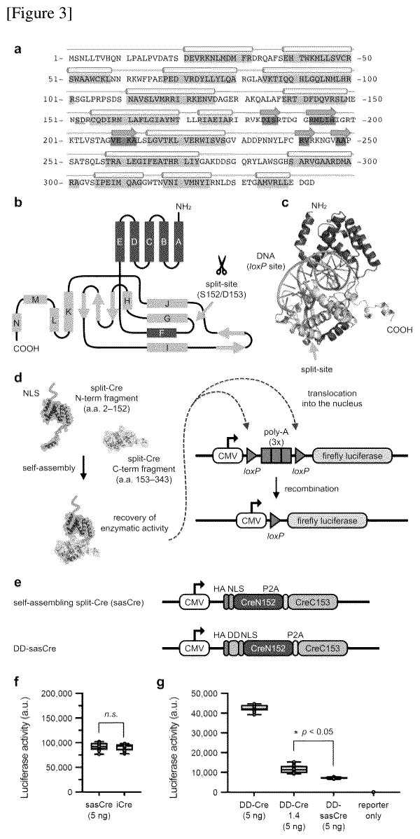

Moreover, several studies have been reported that split Cre recombinase fragments spontaneously recovers its original enzymatic activity by self-assembling (Jullien et al., Nucleic Acids Res. 31: e131 2003: Weinberg et al., Nat. Commun. 10:4845, 2019). Thus, Cre recombinase was split into two fragments at the split site between Ser152 and Asp153 in the C-terminal globe (FIG. 3a, b, and c). In order to confirm the self-assembling ability of the two split fragments, each fragment was allowed to express in HEK293 cells, using a P2A self-cleaving peptide sequence (FIGS. 3d and e). As a result, the split-Cre consisting of the N-terminal fragment (a.a. 2-152) and the C-terminal fragment (a.a. 153-343) exhibited a high recombination efficiency comparable to that of iCre (FIG. 3f).

From these results, it was confirmed that split Cre has self-assembling ability and recovers its enzyme activity by assembling. Hereinafter, self-assembling split Cre is referred to as sasCre (self-assembling split-Cre).

Next, whether or not sasCre tagged with a single DD sequence on the N terminal side of Cre (i.e. DD-sasCre) causes background DNA recombination activity (leakage of activity) in the absence of TMP was examined (FIG. 3e). As a result, as shown in FIG. 3g, DD-sasCre had significantly lower activity leakage than DD-Cre 1.4.

These results suggest that the approach of downsizing Cre recombinase by splitting into fragments is extremely effective to improve its protein degradation efficiency induced by the DD sequence.

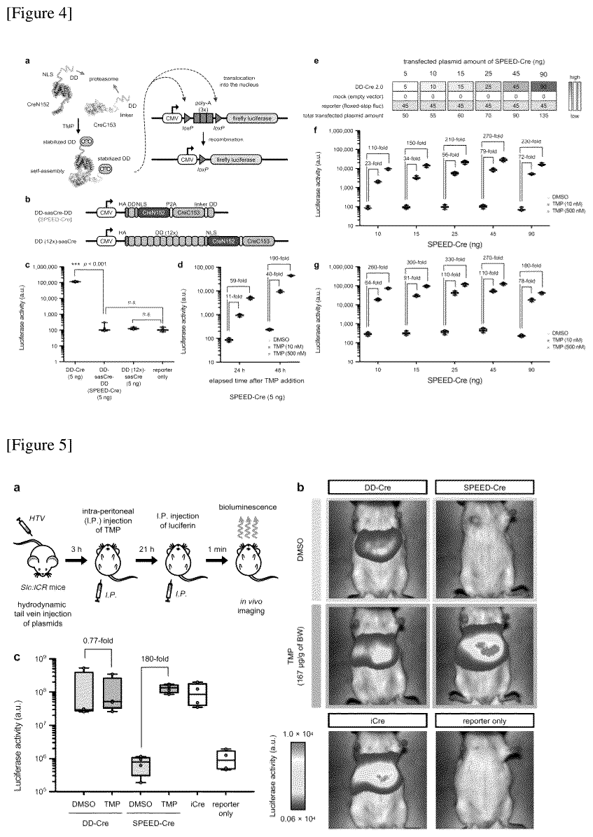

In order to further suppress the leakage of the recombination activity of DD-sasCre, a single DD sequence was tagged to the C-terminus of Cre (FIGS. 4a and b). As a result, the leakage of the background recombination activity of DD-sasCre-DD became an extremely low level that was comparable to the case of using the pcDNA3.1 empty vector plasmid and the reporter plasmid (FIG. 4c). The similar results were observed in sasCre tagged with twelve DD sequences (FIGS. 4b and c). Further, DD-sasCre-DD not only had low background recombination activity, but also exhibited extremely high TMP-dependent DNA recombination activity (not less than 10-fold) (FIG. 4d). Hereinafter, the above-described DD-sasCre-DD is referred to as SPEED-Cre.

The relationship between the expression level of SPEED-Cre and the leakage of DNA recombination activity was examined (FIG. 4e). As a result, it became clear that the DNA recombination activity of SPEED-Cre was maintained a low level in the absence of TMP, even in a case where the amount of the expression vector of SPEED-Cre introduced into the cells was increased to 10 times (FIGS. 4f and g). On the other hand, in the presence of TMP, SPEED-Cre exhibited extremely high TMP-dependent DNA recombination activity (up to about 330-fold) (FIGS. 4f and g). These results indicate that SPEED-Cre is a very powerful and useful tool capable of controlling Cre-loxP recombination in a chemical substance-dependent manner, without causing the leakage of the activity.

### 2-4. Evaluation of Properties of SPEED-Cre In Vivo

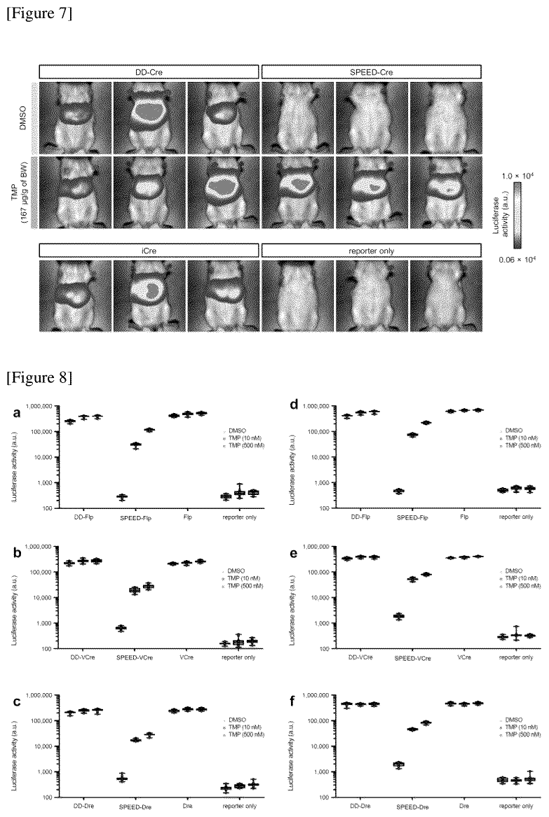

Next, the usefulness of SPEED-Cre in a living mouse body, namely, in vivo was examined. Using a hydrodynamic tail vain (HTV) injection method (Liu et al., Gene Ther. 6:1258-1266, 1999), mice liver was we transiently transfected with DD-Cre or SPEED-Cre (FIG. 5a). Three hours after the HTV injection, TMP (167 μg/g body weight) or DMSO was intraperitoneally (I.P.) administered into the DD-Cre- or SPEED-Cre-transfected mice (FIG. 5a). The DNA recombination activity was measured using the bioluminescence of a luciferase reporter as an indicator. As a result, in the absence of TMP, DD-Cre had high DNA recombination activity at the same level as iCre, and it became clear that it caused the leakage of the activity (FIGS. 5b and c, and FIG. 7). Further, even if the mice were treated with TMP at a concentration of 167 μg/g body weight, DNA recombination was not induced (FIGS. 5b and c, and FIG. 7). The DNA recombination ability of DD-Cre in vivo was consistent to the ability in in vitro experiments using the human cell line. On the contrast, in SPEED-Cre, the leakage level of background DNA recombination activity was suppressed to low, comparable to a negative control group using the pcDNA3.1 empty vector plasmid (FIGS. 5b and c, and FIG. 8). In addition, SPEED-Cre had an ability to exhibit high TMP-dependent DNA recombination activity, comparable to the recombination activity of Cre (FIGS. 5b and c, and FIG. 7).

These results indicate that SPEED-Cre efficiently induces chemically inducible Cre-loxP recombination without causing the leakage of DNA recombination activity in vivo.

### 5. Application of Method of Producing SPEED-Cre to Other Proteins

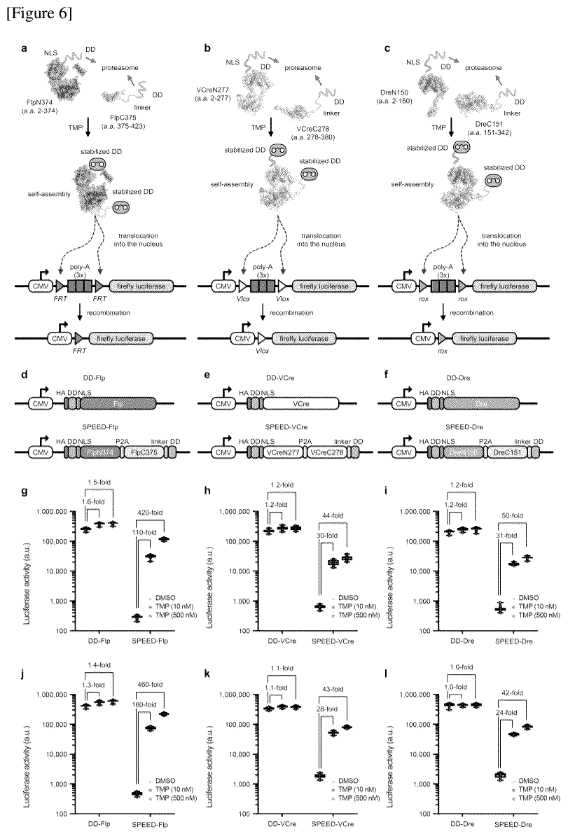

Whether or not the method for producing SPEED-Cre could be applied to other proteins was examined. Specifically, Flp-FRT, VCre-Vlox and Dre-rox that are representative recombination systems with tyrosine-type site-specific DNA recombinases, as with Cre, were examined. These recombination systems have been widely used, as with Cre-loxP. Moreover, Flp, VCre and Dre recombinases have an already known split site that can spontaneously recover its enzymatic activity by self-assembling the two fragments (Weinberg et al., Nat. Commun. 10:4845, 2019). Thus, similar to SPEED-Cre, a single DD sequence was tagged into the N-terminal and the C-terminal sides of each recombinase, and the recombinases were then split into two fragments to generate SPEED-Flp, SPEED-VCre and SPEED-Dre (FIG. 6a to f). As expected, SPEED-Flp only showed an extremely low background level of DNA recombination activity comparable to a negative control group using the pcDNA3.1 empty vector plasmid, and induced extremely high TMP-dependent DNA recombination activity (FIGS. 6g and j, and FIGS. 8a and d). On the contrast, as with DD-Cre, Flp tagged with single DD (DD-Flp) caused the leakage of high background DNA recombination activity comparable to full-length Flp in the absence of TMP (FIGS. 6g and j, and FIGS. 8a and d).

The results similar to those of SPEED-Cre were observed in SPEED-VCre (FIGS. 6h and k, and FIGS. 8b and e) and SPEED-Dre (FIGS. 6i and 1, and FIGS. 8c and f).

These results indicate that the method for producing SPEED-Cre can be applied to other proteins.

### II. Analysis of SPEED-Cas9

### 1. Methods

### 1-1. Reagents, Etc.

The oligonucleotides used for construction of plasmids were synthesized by Eurofines Genomics. The mammalian expression vector, pcDNA 3.1/V 5-HisA, was purchased from ThermoFisher Scientific. The promoterless vector for construction of a donor plasmid, pCold-I, was purchased from Clontech. All plasmids were constructed based on individual methods, namely, an overlapping PCR extension method, the cleavage of a PCR product with restriction enzymes, and ligation of double-stranded DNA with T4 DNA ligase. The PCR reaction was performed using DNA polymerase of KOD One PCR Master Mix (TOYOBO) according to the manufacturer's instructions. All restriction enzymes were obtained from New England bioLabs, and were used according to the manufacturer's instructions. As T4 ligase, Ligation high Ver.2 (TOYOBO) was used. All the constructed plasmids were checked by DNA sequencing analysis service (Eurofines Genomics).

### 1-2. Construction of Plasmids

For constructing destabilized Cas9, sequences encoding a degradation domain (DD) derived from E. coli dihydrofolate reductase (ecDHFR), a nuclear localization signal (NLS), a P2A self-cleaving 2A peptide, hetero-leucine zipper NZ, hetero-leucine zipper CZ, or spCas9 endonuclease encoded by codons optimized for mammalian expression were synthesized by Eurofines Genomics. The sequence encoding spCas9 endonuclease was inserted into the HindIII-XbaI sites in a pcDNA 3.1/V 5-HisA vector having a CMV promoter.

For constructing a luciferase reporter for detecting Cas9 endonuclease activity, the sequence encoding firefly luciferase (Fluc) was amplified from pGL4.31 vector (Promega). The firefly luciferase with an in-flamed stop codon (StopFluc) was produced by an overlapping PCR extension method. The primer (5′-aacttgcacgagatctaaagcggcggggcgccg-3′: SEQ ID No: 40) was used for PCR. The Stop-Fluc construct was inserted into the HindII-XhoI site of pcDNA 3.1/V 5-HisA having the CMV promoter. A luciferase donor vector was constructed by inserting the reverse firefly luciferase sequence into the XhoI and HindIII site of the bacterial expression pCold-I vector (Clontech), and was used as donor DNA.

### 1-3. Cell Culture

HEK293T cells (American Type Culture Collection: ATCC) were cultured at 37° C. under 5% CO₂ in Dulbecco's Modified Eagle Medium (DMEM: Thermo Fisher Scientific) supplemented with 10% fetal bovine serum (FBS: Thermo Fisher Scientific), 100 units/ml of penicillin and 100 μg/ml of streptomycin (Thermo Fisher Scientific) and 2 mM GlutaMAX (Thermo Fisher Scientific).

### 1-4. Site-Specific Recombination Experiments In Vitro

For luciferase assay, HEK 293 cells were plated at 25,000 cells per well on a 96-well flat clear-bottom microplate (Greiner Bio-One), and were then cultured for 24 hours at 37° C. in 5% CO₂. The cells were transfected with cDNAs encoding destabilized Cas9 or an empty vector (negative control) along with the luciferase reporter (StopFluc), donor DNA (Fluc) and Cas9 encoding gRNA, using Lipofectamine 3000 (Thermo Fisher Scientific) at 37° C. The total amount of DNA was 50 ng per well. The transfection ratio of the destabilized Cas9, the luciferase reporter, the donor DNA, and the gRNA was set to be equal (1:1:1:1). Twenty-four hours after the transfection, TMP or DMSO was added into the cells, and the cells were further cultured for 48 hours. Just before the luciferase assay, the medium was replaced with Hanks' balanced salt solution (HBSS) (Thermo Fisher Scientific) containing 1 mM D-luciferin potassium salt. Bioluminescence measurement was performed for 1 second per well at room temperature using a Centro XS³ LB 960 plate-reading luminometer (Berthold Technologies).

Full-length Cas9 was used as a positive control. The transfection ratio of the full-length Cas9, the luciferase reporter, the donor DNA, and the gRNA was set to be equal (1:1:1:1). Twenty-four hours after the transfection, TMP or DMSO was added into the cells, and the cells were further cultured for 24 hours. Just before the luciferase assay, the medium was replaced with Hanks' balanced salt solution (HBSS) (Thermo Fisher Scientific) containing 1 mM D-luciferin potassium salt. Bioluminescence measurement was performed for 1 second per well at room temperature using a Centro XS³ LB 960 plate-reading luminometer (Berthold Technologies).

### 1-5. Reproducibility and Statistics Analysis

No power studies were performed in cell culture experiments. None of samples were excluded from analysis. A method of randomization was performed in the cell culture experiments. No blinding was used in the cell culture experiments.

### 2. Results

### 2-1. Application of Method for Producing SPEED-Cre to Other Proteins

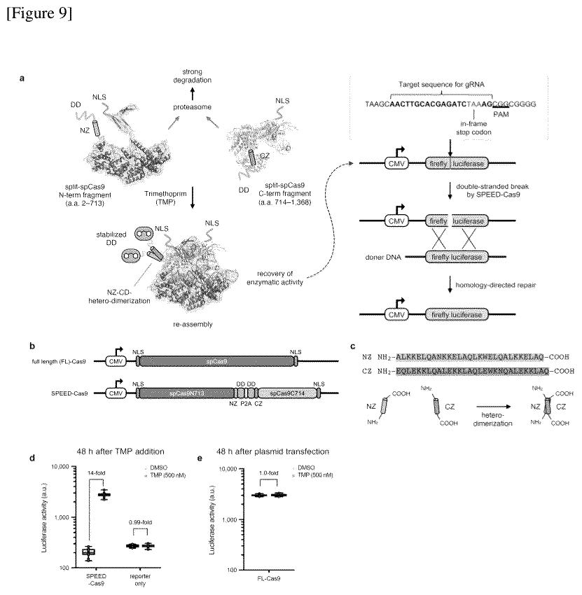

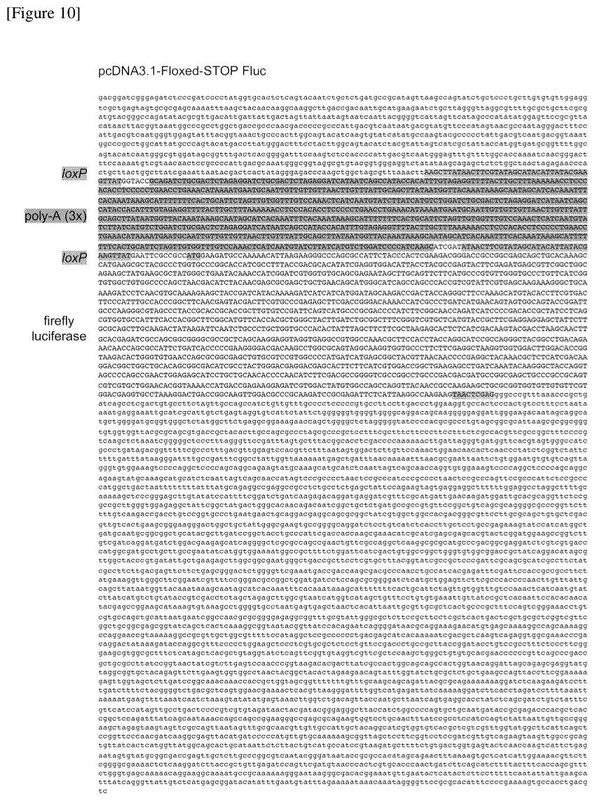

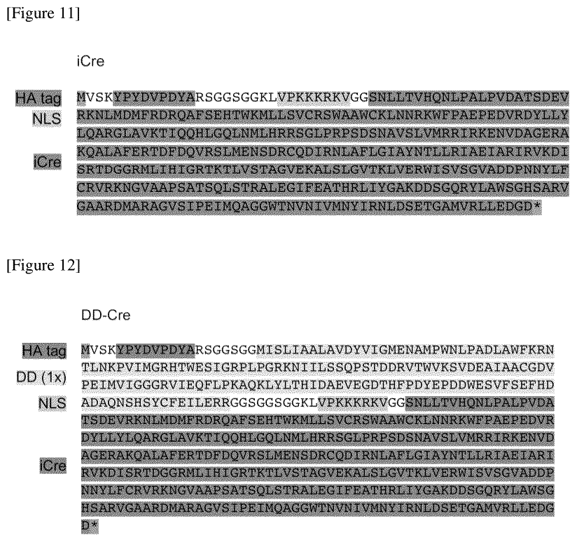

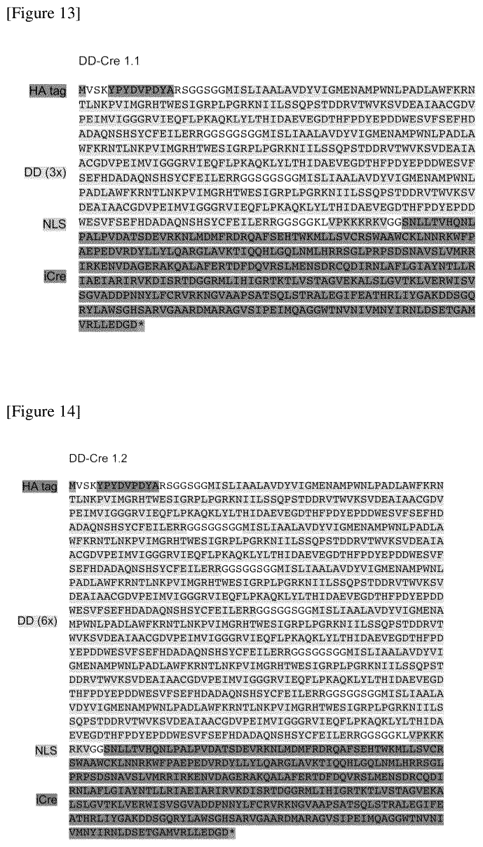

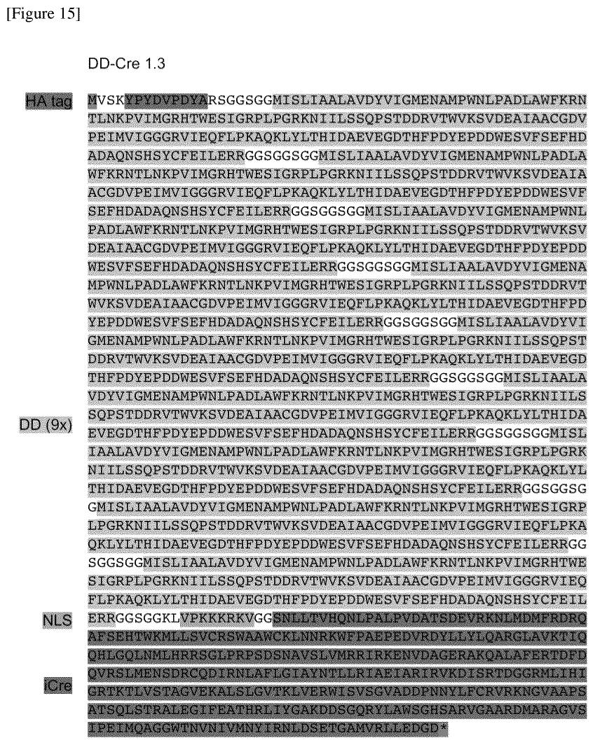

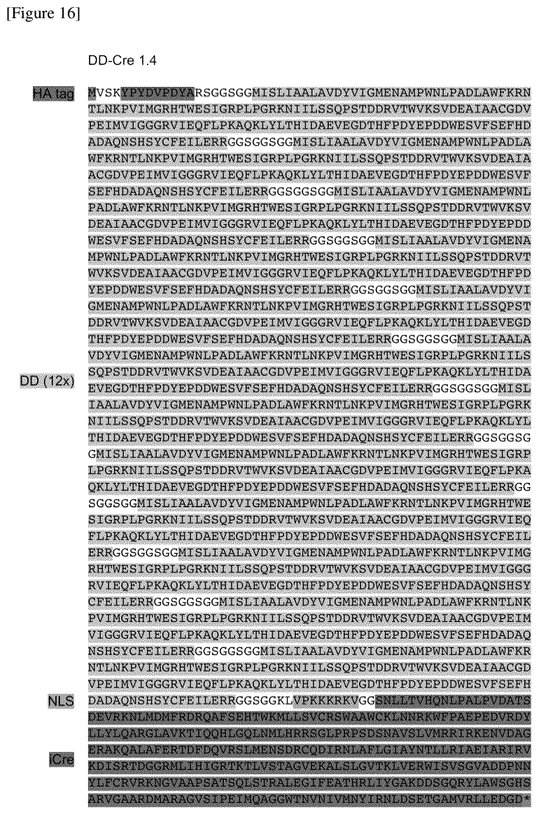

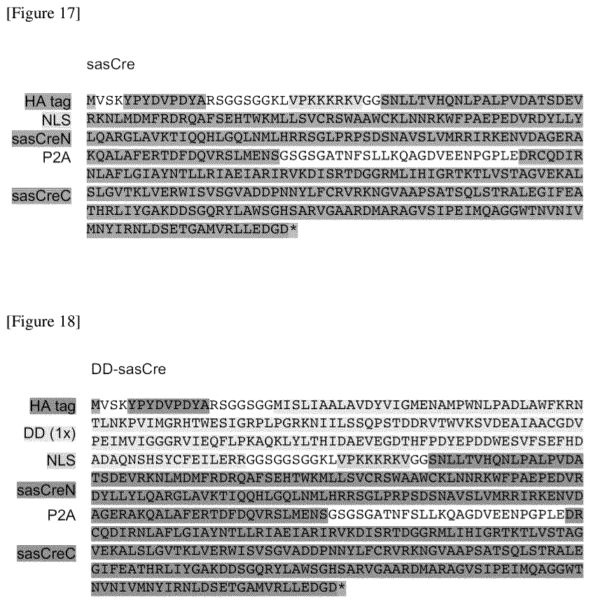

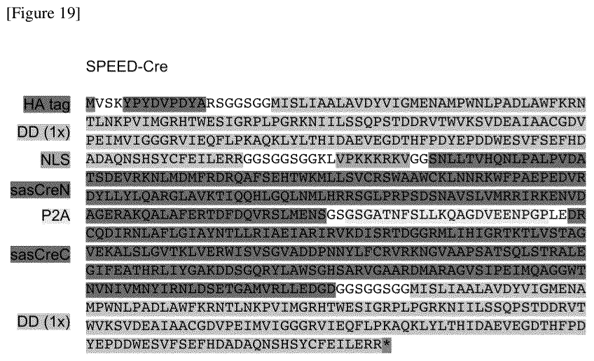

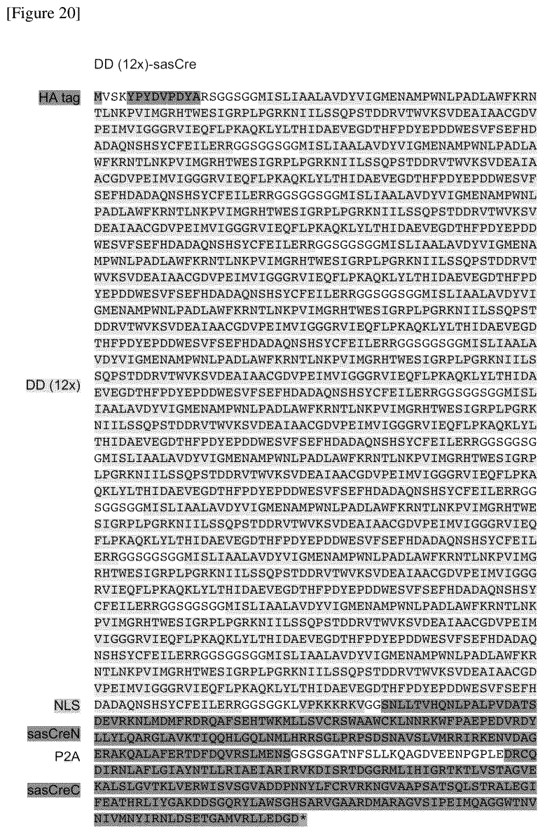

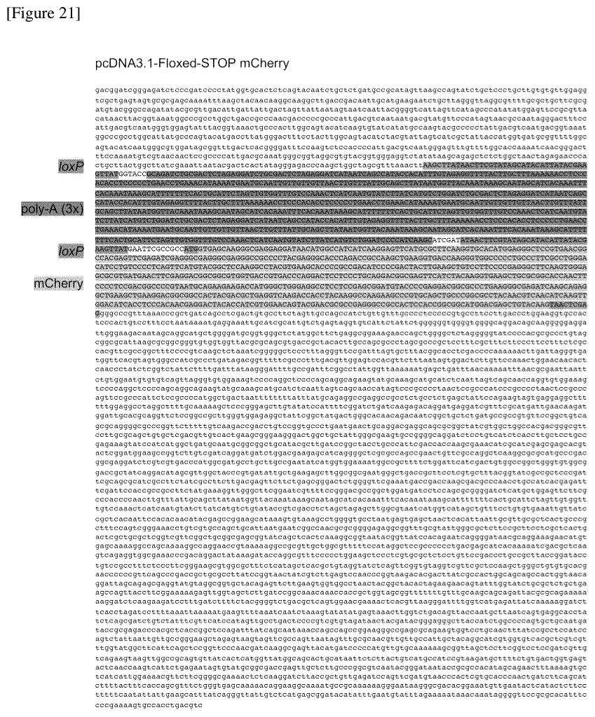

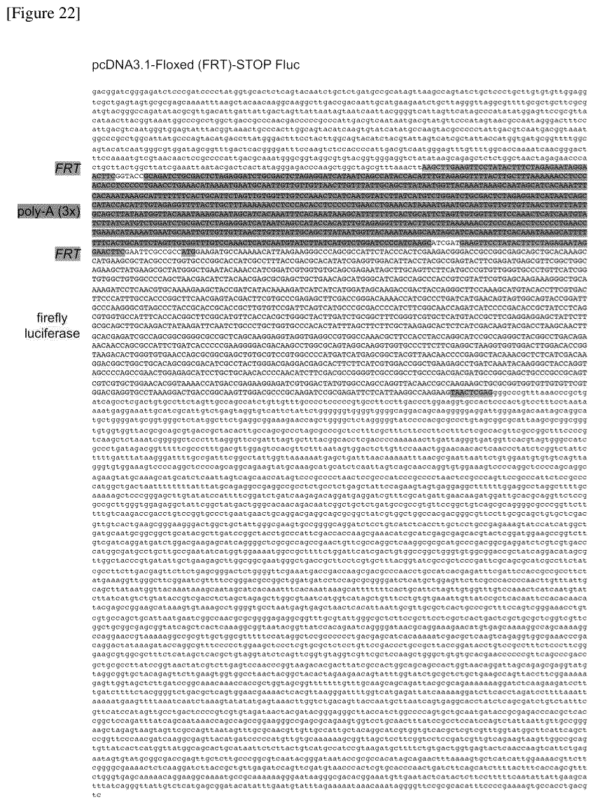

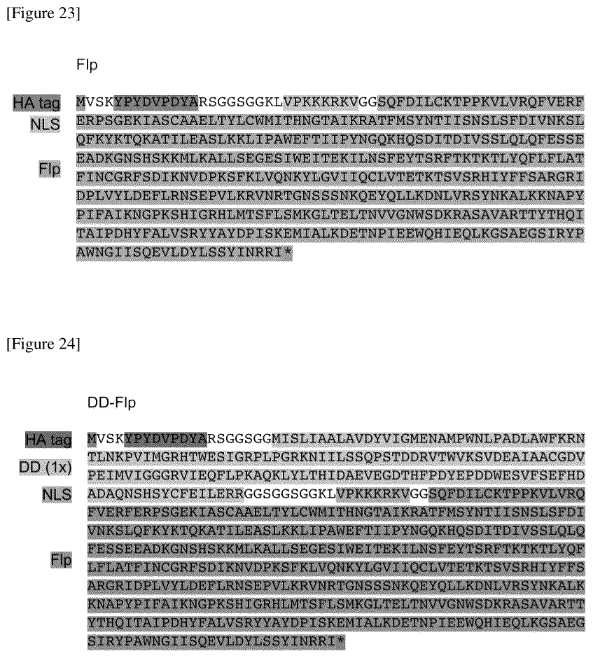

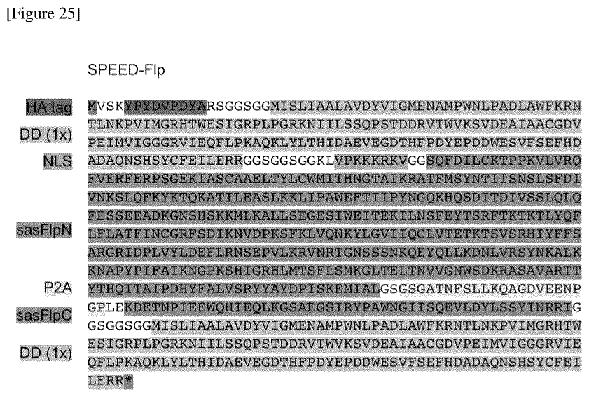

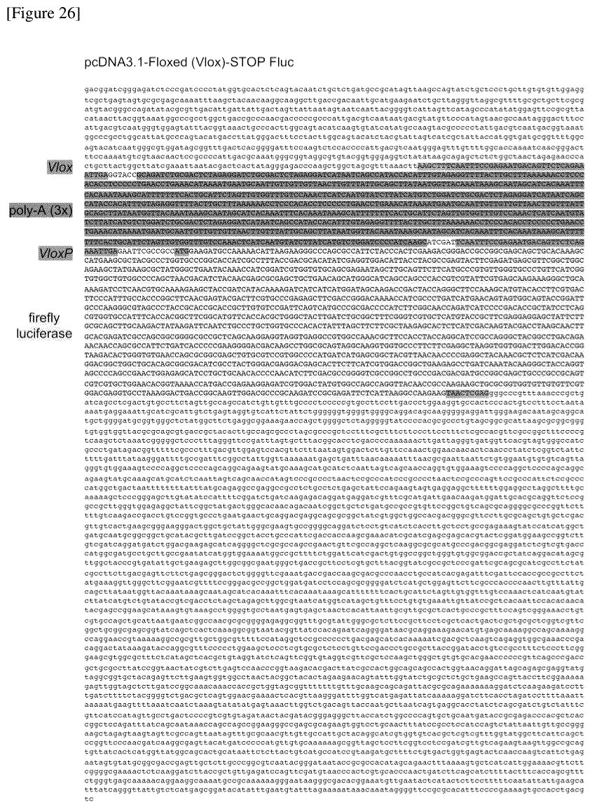

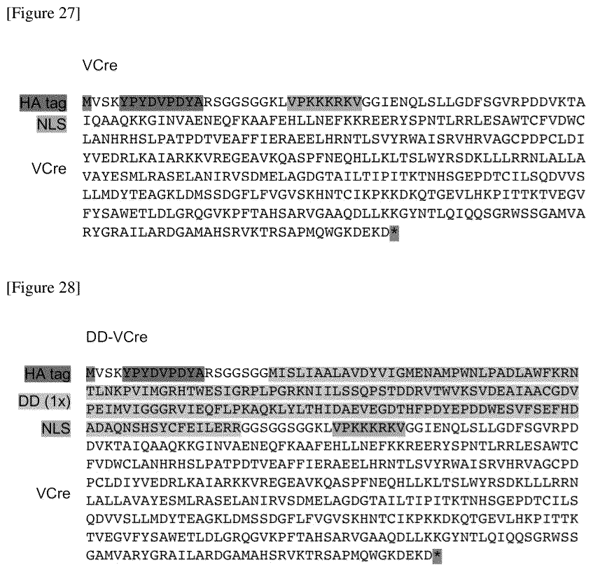

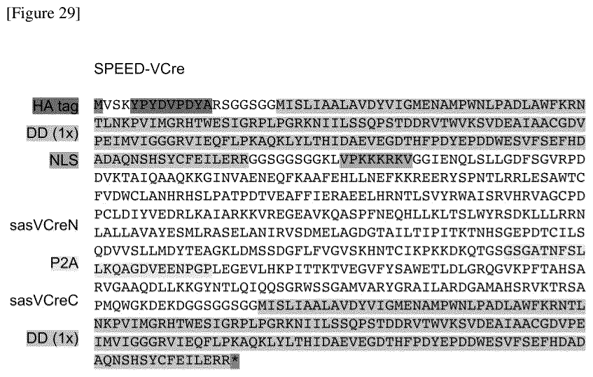

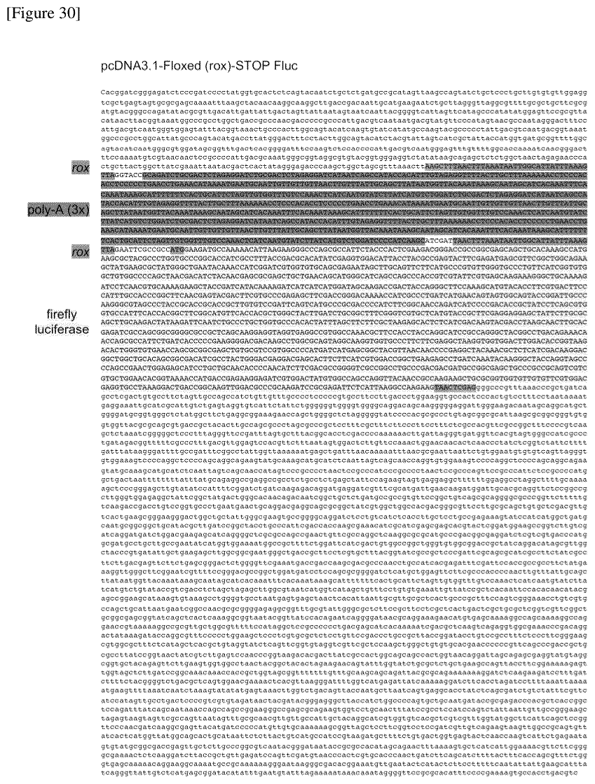

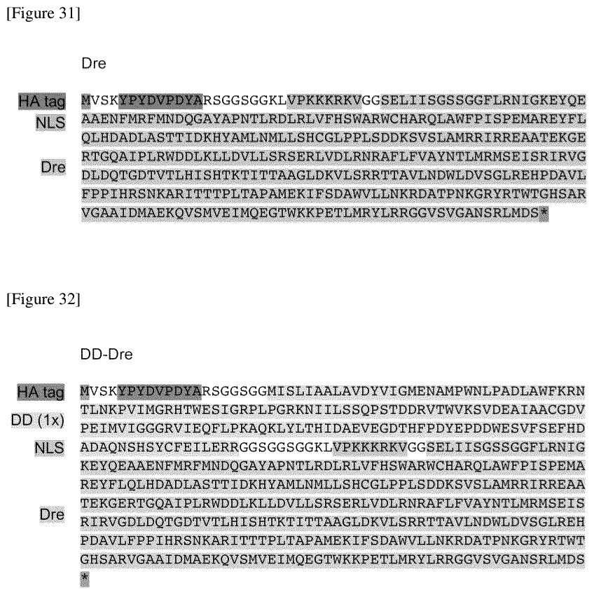

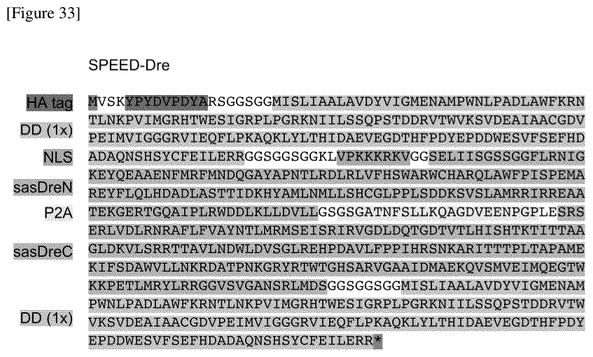

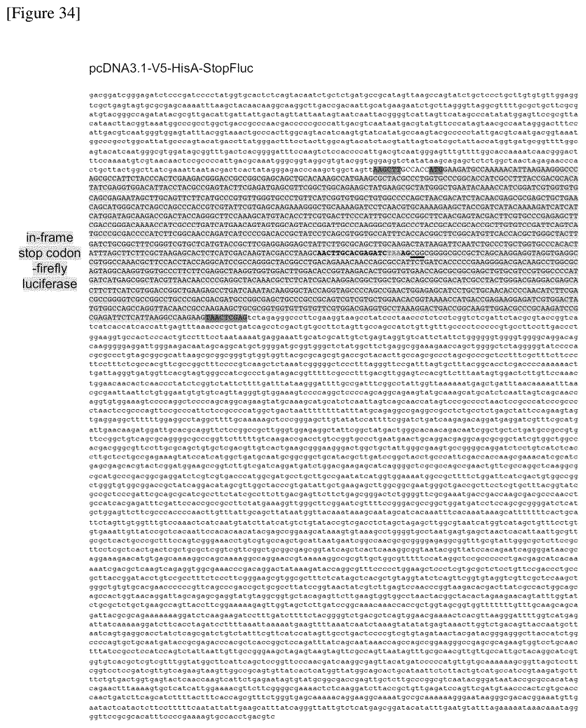

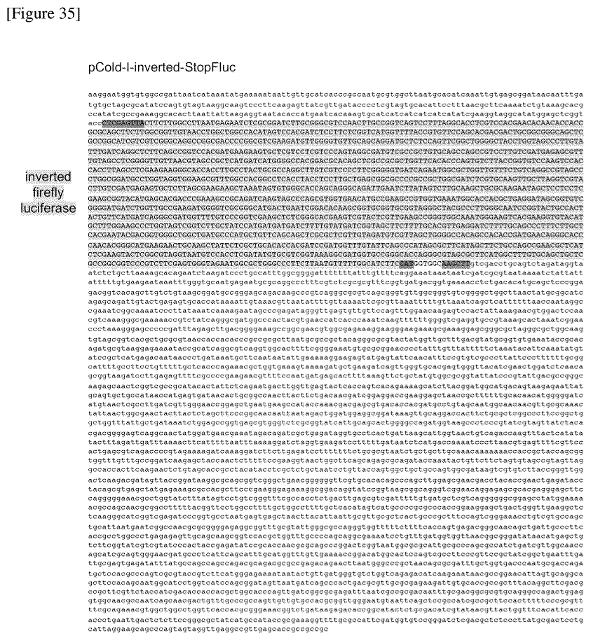

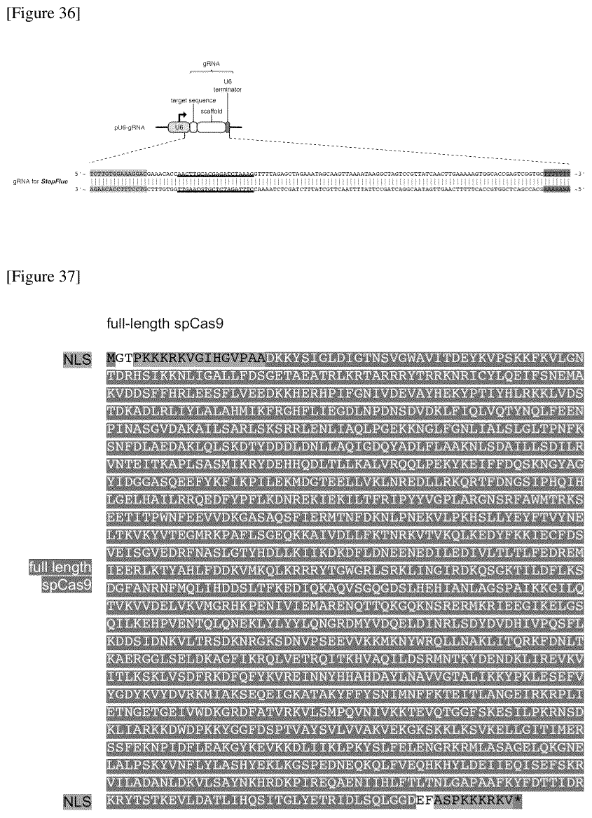

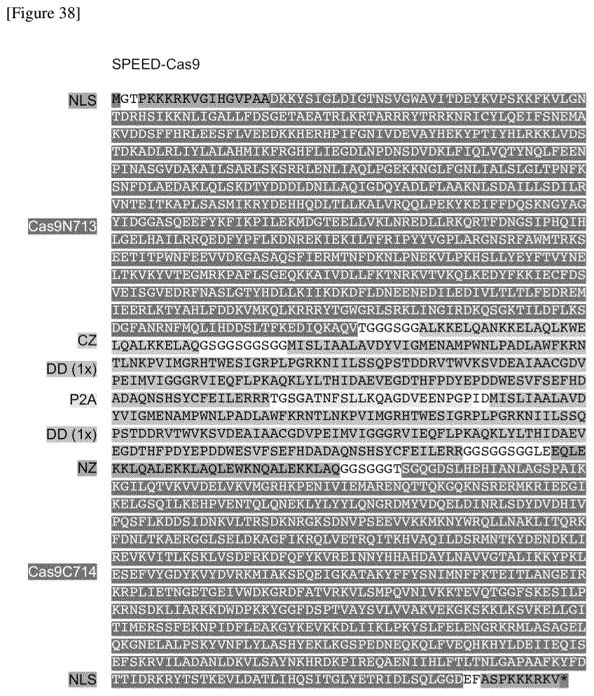

Whether or not the method for producing SPEED-Cre could be applied to other proteins that have a large molecular size and do not self-assemble was examined. Specifically, Cas9, a Streptococcus pyogenes-derived endonuclease, which is about four times larger in molecular size than Cre recombinase and is a representative tool for genome editing technology, was examined. Regarding SpCas9, a split site has already been known, in which when SpCas9 is fused with a photoswitch protein and is then dimerized in a light-dependent manner, so that two fragments are put in close proximity, structural complementation occurs and the enzyme activity is recovered (Nihongaki et al. Biotechnol. 33:755-760, 2015). Thus, Cas9 endonuclease was split into two fragments at the split site between Val713 and Ser714 (FIGS. 9a and b). As a dimerizer, the hetero-leucine zipper pair NZ and CZ (Ghosh et al., JACS 122:5668-5659, 2000) was utilized instead of the photoswitch protein (FIGS. 9a, b and c). NZ and a DD sequence were tagged into the C-terminus of the N-terminal fragment (a.a. 2-713) of the split Cas9 endonuclease, and CZ and a DD sequence were tagged into the N-terminus of the C-terminal fragment (a.a. 714-1,368) of the split Cas9 endonuclease, and then, both fragments were fused with each other via P2A to produce SPEED-Cas9 (FIGS. 9a and b). Surprisingly, SPEED-Cas9 showed only an extremely low background level of endonuclease activity, comparable to that of the negative control group using the pcDNA3.1 empty vector plasmid, and induced extremely high TMP-dependent endonuclease activity (FIG. 9d). Moreover, the TMP-dependent endonuclease activity of SPEED-Cas9 was comparable to that of full-length Cas9 (FIGS. 9d and e). Similar to SPEED-Cre, the results involving an extremely low background level in the absence of TMP and high TMP-dependent induction efficiency were also observed in SPEED-Cas9.

The above-described results indicate that the method for producing SPEED-Cre can be applied to other proteins, in particular, to proteins with extremely large molecular sizes, and even, to non-self-assembling split proteins.

### INDUSTRIAL APPLICABILITY

According to the present invention, a method of controlling the activity of a protein using a chemical substance is provided. Therefore, through the control of the activity of a protein, the present method can be used for the purpose of regulating cellular functions, and further, can be used for the purpose of treating diseases, etc. The present method is expected to be utilized in the fields of medicine, pharmacy, etc.

1. A plurality of protein fragments, which recover the activity of the protein when the plurality of protein fragments assemble, and in which each of the plurality of protein fragments is tagged with a degradation domain (DD) sequence.

2. The plurality of protein fragments according to claim 1, wherein each fragment of the protein fragments has a binding region to one or multiple fragments other than the fragment.

3. The plurality of protein fragments according to claim 2, wherein the binding region is an exogenous peptide.

4. The plurality of protein fragments according to claim 3, wherein the exogenous peptide is a leucine zipper peptide or a dimer protein.

5. The plurality of protein fragments according to claim 1, wherein the number of the plurality of protein fragments is 2.

6. The plurality of protein fragments according to claim 1, wherein the protein is any one of a genome editing technology-related protein, a transcription factor-based protein, a reporter-based protein, a reprogramming-related protein, and a cell death-related protein.

7. The plurality of protein fragments according to claim 1, wherein the DD sequence is any one of a DHFR-derived sequence, an FKBP-derived sequence, an FKBP12 mutant-derived sequence, and a UnaG-derived sequence.

8. A nucleic acid encoding each fragment of the plurality of protein fragments according to claim 1.

9. An expression vector having the nucleic acid according to claim 8.

10. A genome editing kit, comprising
the plurality of protein fragments according to claim 1 and/or the expression vector comprising a nucleic acid encoding each of the plurality of protein fragments.

11. The kit according to claim 10, wherein the plurality of protein fragments are site-specific recombinases, TALE proteins, or Cas9 nucleases.

12. The kit according to claim 10, wherein the expression vector has comprises a nucleic acid encoding each of the plurality of protein fragments that are site-specific recombinases, TALE proteins, or Cas9 nucleases.

13. A method of regulating the activity of a protein in a cell, comprising:
introducing into a cell, a fusion of a degradation domain (DD) sequence, and each fragment of a protein that has been split into fragments, such that the fragments recover the activity of the protein when they assemble; and
introducing a DD sequence-specific stabilizing factor into the cell.

14. The method according to claim 13, wherein the protein is any one of a genome editing technology-related protein, a transcription factor-based protein, a reporter-based protein, a reprogramming-related protein, and a cell death-related protein.

15. The method according to claim 13, wherein the DD sequence is any one of a DHFR-derived sequence, an FKBP-derived sequence, an FKBP12 mutant-derived sequence, and a UnaG-derived sequence.


SEQUENCE LISTING (40 sequences)

SEQ ID NO: 1 - DNA, 7877 bp, synthetic construct
  misc_feature 1..7877: note = Reporter gene expression cassette
  misc_feature 917..950: note = loxP site
  polyA_site 957..1752
  misc_feature 1759..1792: note = loxP sequence
  gene 1808..3436
  gene 1808..3436: note = Firefly luciferase
  source 1..7877: mol_type = other DNA
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttataacttcgtatagcatacattatacgaagttatggtaccgcagatctgcgactctagaggatctgcgactctagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgcgactctagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgcgactctagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatccccatcaagcatcgatataacttcgtatagcatacattatacgaagttatgaattcgccgccatggaagatgccaaaaacattaagaagggcccagcgccattctacccactcgaagacgggaccgccggcgagcagctgcacaaagccatgaagcgctacgccctggtgcccggcaccatcgcctttaccgacgcacatatcgaggtggacattacctacgccgagtacttcgagatgagcgttcggctggcagaagctatgaagcgctatgggctgaatacaaaccatcggatcgtggtgtgcagcgagaatagcttgcagttcttcatgcccgtgttgggtgccctgttcatcggtgtggctgtggccccagctaacgacatctacaacgagcgcgagctgctgaacagcatgggcatcagccagcccaccgtcgtattcgtgagcaagaaagggctgcaaaagatcctcaacgtgcaaaagaagctaccgatcatacaaaagatcatcatcatggatagcaagaccgactaccagggcttccaaagcatgtacaccttcgtgacttcccatttgccacccggcttcaacgagtacgacttcgtgcccgagagcttcgaccgggacaaaaccatcgccctgatcatgaacagtagtggcagtaccggattgcccaagggcgtagccctaccgcaccgcaccgcttgtgtccgattcagtcatgcccgcgaccccatcttcggcaaccagatcatccccgacaccgctatcctcagcgtggtgccatttcaccacggcttcggcatgttcaccacgctgggctacttgatctgcggctttcgggtcgtgctcatgtaccgcttcgaggaggagctattcttgcgcagcttgcaagactataagattcaatctgccctgctggtgcccacactatttagcttcttcgctaagagcactctcatcgacaagtacgacctaagcaacttgcacgagatcgccagcggcggggcgccgctcagcaaggaggtaggtgaggccgtggccaaacgcttccacctaccaggcatccgccagggctacggcctgacagaaacaaccagcgccattctgatcacccccgaaggggacgacaagcctggcgcagtaggcaaggtggtgcccttcttcgaggctaaggtggtggacttggacaccggtaagacactgggtgtgaaccagcgcggcgagctgtgcgtccgtggccccatgatcatgagcggctacgttaacaaccccgaggctacaaacgctctcatcgacaaggacggctggctgcacagcggcgacatcgcctactgggacgaggacgagcacttcttcatcgtggaccggctgaagagcctgatcaaatacaagggctaccaggtagccccagccgaactggagagcatcctgctgcaacaccccaacatcttcgacgccggggtcgccggcctgcccgacgacgatgccggcgagctgcccgccgcagtcgtcgtgctggaacacggtaaaaccatgaccgagaaggagatcgtggactatgtggccagccaggttacaaccgccaagaagctgcgcggtggtgttgtgttcgtggacgaggtgcctaaaggactgaccggcaagttggacgcccgcaagatccgcgagattctcattaaggccaagaagtaactcgaggggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 2 - protein, 374 aa, Bacteriophage P1
  REGION 5..13: note = MISC_FEATURE - HAtag
  SIGNAL 23..30
  SIGNAL 23..30: note = Nuclear Localization Signal
MVSKYPYDVPDYARSGGSGGKLVPKKKRKVGGSNLLTVHQNLPALPVDATSDEVRKNLMDMFRDRQAFSEHTWKMLLSVCRSWAAWCKLNNRKWFPAEPEDVRDYLLYLQARGLAVKTIQQHLGQLNMLHRRSGLPRPSDSNAVSLVMRRIRKENVDAGERAKQALAFERTDFDQVRSLMENSDRCQDIRNLAFLGIAYNTLLRIAEIARIRVKDISRTDGGRMLIHIGRTKTLVSTAGVEKALSLGVTKLVERWISVSGVADDPNNYLFCRVRKNGVAAPSATSQLSTRALEGIFEATHRLIYGAKDDSGQRYLAWSGHSARVGAARDMARAGVSIPEIMQAGGWTNVNIVMNYIRNLDSETGAMVRLLEDGD

SEQ ID NO: 3 - protein, 541 aa, Bacteriophage P1
  REGION 5..13: note = MISC_FEATURE - HAtag
  DOMAIN 21..179: note = degradation domain
  SIGNAL 190..197: note = Nuclear Localization Signal
MVSKYPYDVPDYARSGGSGGMISLIAALAVDYVIGMENAMPWNLPADLAWFKRNTLNKPVIMGRHTWESIGRPLPGRKNIILSSQPSTDDRVTWVKSVDEAIAACGDVPEIMVIGGGRVIEQFLPKAQKLYLTHIDAEVEGDTHFPDYEPDDWESVFSEFHDADAQNSHSYCFEILERRGGSGGSGGKLVPKKKRKVGGSNLLTVHQNLPALPVDATSDEVRKNLMDMFRDRQAFSEHTWKMLLSVCRSWAAWCKLNNRKWFPAEPEDVRDYLLYLQARGLAVKTIQQHLGQLNMLHRRSGLPRPSDSNAVSLVMRRIRKENVDAGERAKQALAFERTDFDQVRSLMENSDRCQDIRNLAFLGIAYNTLLRIAEIARIRVKDISRTDGGRMLIHIGRTKTLVSTAGVEKALSLGVTKLVERWISVSGVADDPNNYLFCRVRKNGVAAPSATSQLSTRALEGIFEATHRLIYGAKDDSGQRYLAWSGHSARVGAARDMARAGVSIPEIMQAGGWTNVNIVMNYIRNLDSETGAMVRLLEDGD

SEQ ID NO: 4 - protein, 872 aa, Bacteriophage P1
  REGION 5..13: note = MISC_FEATURE - HAtag
  DOMAIN 21..179: note = Degradation Domain
  DOMAIN 188..346: note = Degradation Domain
  DOMAIN 355..513: note = Degradation Domain
  SIGNAL 521..528: note = Nuclear Localization Signal
MVSKYPYDVPDYARSGGSGGMISLIAALAVDYVIGMENAMPWNLPADLAWFKRNTLNKPVIMGRHTWESIGRPLPGRKNIILSSQPSTDDRVTWVKSVDEAIAACGDVPEIMVIGGGRVIEQFLPKAQKLYLTHIDAEVEGDTHFPDYEPDDWESVFSEFHDADAQNSHSYCFEILERRGGSGGSGGMISLIAALAVDYVIGMENAMPWNLPADLAWFKRNTLNKPVIMGRHTWESIGRPLPGRKNIILSSQPSTDDRVTWVKSVDEAIAACGDVPEIMVIGGGRVIEQFLPKAQKLYLTHIDAEVEGDTHFPDYEPDDWESVFSEFHDADAQNSHSYCFEILERRGGSGGSGGMISLIAALAVDYVIGMENAMPWNLPADLAWFKRNTLNKPVIMGRHTWESIGRPLPGRKNIILSSQPSTDDRVTWVKSVDEAIAACGDVPEIMVIGGGRVIEQFLPKAQKLYLTHIDAEVEGDTHFPDYEPDDWESVFSEFHDADAQNSHSYCFEILERRGGSGGKLVPKKKRKVGGSNLLTVHQNLPALPVDATSDEVRKNLMDMFRDRQAFSEHTWKMLLSVCRSWAAWCKLNNRKWFPAEPEDVRDYLLYLQARGLAVKTIQQHLGQLNMLHRRSGLPRPSDSNAVSLVMRRIRKENVDAGERAKQALAFERTDFDQVRSLMENSDRCQDIRNLAFLGIAYNTLLRIAEIARIRVKDISRTDGGRMLIHIGRTKTLVSTAGVEKALSLGVTKLVERWISVSGVADDPNNYLFCRVRKNGVAAPSATSQLSTRALEGIFEATHRLIYGAKDDSGQRYLAWSGHSARVGAARDMARAGVSIPEIMQAGGWTNVNIVMNYIRNLDSETGAMVRLLEDGD

SEQ ID NO: 5 - protein, 1373 aa, Bacteriophage P1
  REGION 5..13: note = MISC_FEATURE - HAtag
  DOMAIN 21..179: note = Degradation Domain
  DOMAIN 188..346: note = Degradation Domain
  DOMAIN 355..513: note = Degradation Domain
  DOMAIN 522..680: note = Degradation Domain
  DOMAIN 689..847: note = Degradation Domain
  DOMAIN 856..1014: note = Degradation Domain
  SIGNAL 1022..1029: note = Nuclear Localization Signal
MVSKYPYDVPDYARSGGSGGMISLIAALAVDYVIGMENAMPWNLPADLAWFKRNTLNKPVIMGRHTWESIGRPLPGRKNIILSSQPSTDDRVTWVKSVDEAIAACGDVPEIMVIGGGRVIEQFLPKAQKLYLTHIDAEVEGDTHFPDYEPDDWESVFSEFHDADAQNSHSYCFEILERRGGSGGSGGMISLIAALAVDYVIGMENAMPWNLPADLAWFKRNTLNKPVIMGRHTWESIGRPLPGRKNIILSSQPSTDDRVTWVKSVDEAIAACGDVPEIMVIGGGRVIEQFLPKAQKLYLTHIDAEVEGDTHFPDYEPDDWESVFSEFHDADAQNSHSYCFEILERRGGSGGSGGMISLIAALAVDYVIGMENAMPWNLPADLAWFKRNTLNKPVIMGRHTWESIGRPLPGRKNIILSSQPSTDDRVTWVKSVDEAIAACGDVPEIMVIGGGRVIEQFLPKAQKLYLTHIDAEVEGDTHFPDYEPDDWESVFSEFHDADAQNSHSYCFEILERRGGSGGSGGMISLIAALAVDYVIGMENAMPWNLPADLAWFKRNTLNKPVIMGRHTWESIGRPLPGRKNIILSSQPSTDDRVTWVKSVDEAIAACGDVPEIMVIGGGRVIEQFLPKAQKLYLTHIDAEVEGDTHFPDYEPDDWESVFSEFHDADAQNSHSYCFEILERRGGSGGSGGMISLIAALAVDYVIGMENAMPWNLPADLAWFKRNTLNKPVIMGRHTWESIGRPLPGRKNIILSSQPSTDDRVTWVKSVDEAIAACGDVPEIMVIGGGRVIEQFLPKAQKLYLTHIDAEVEGDTHFPDYEPDDWESVFSEFHDADAQNSHSYCFEILERRGGSGGSGGMISLIAALAVDYVIGMENAMPWNLPADLAWFKRNTLNKPVIMGRHTWESIGRPLPGRKNIILSSQPSTDDRVTWVKSVDEAIAACGDVPEIMVIGGGRVIEQFLPKAQKLYLTHIDAEVEGDTHFPDYEPDDWESVFSEFHDADAQNSHSYCFEILERRGGSGGKLVPKKKRKVGGSNLLTVHQNLPALPVDATSDEVRKNLMDMFRDRQAFSEHTWKMLLSVCRSWAAWCKLNNRKWFPAEPEDVRDYLLYLQARGLAVKTIQQHLGQLNMLHRRSGLPRPSDSNAVSLVMRRIRKENVDAGERAKQALAFERTDFDQVRSLMENSDRCQDIRNLAFLGIAYNTLLRIAEIARIRVKDISRTDGGRMLIHIGRTKTLVSTAGVEKALSLGVTKLVERWISVSGVADDPNNYLFCRVRKNGVAAPSATSQLSTRALEGIFEATHRLIYGAKDDSGQRYLAWSGHSARVGAARDMARAGVSIPEIMQAGGWTNVNIVMNYIRNLDSETGAMVRLLEDGD

SEQ ID NO: 6 - protein, 1874 aa, Bacteriophage P1
  REGION 5..13: note = MISC_FEATURE - HAtag
  DOMAIN 21..179: note = Degradation Domain
  DOMAIN 188..346: note = Degradation Domain
  DOMAIN 355..513: note = Degradation Domain
  DOMAIN 522..680: note = Degradation Domain
  DOMAIN 689..847: note = Degradation Domain
  DOMAIN 856..1014: note = Degradation Domain
  DOMAIN 1023..1181: note = Degradation Domain
  DOMAIN 1190..1348: note = Degradation Domain
  DOMAIN 1357..1515: note = Degradation Domain
  SIGNAL 1523..1530: note = Nuclear Localization Signal
MVSKYPYDVPDYARSGGSGGMISLIAALAVDYVIGMENAMPWNLPADLAWFKRNTLNKPVIMGRHTWESIGRPLPGRKNIILSSQPSTDDRVTWVKSVDEAIAACGDVPEIMVIGGGRVIEQFLPKAQKLYLTHIDAEVEGDTHFPDYEPDDWESVFSEFHDADAQNSHSYCFEILERRGGSGGSGGMISLIAALAVDYVIGMENAMPWNLPADLAWFKRNTLNKPVIMGRHTWESIGRPLPGRKNIILSSQPSTDDRVTWVKSVDEAIAACGDVPEIMVIGGGRVIEQFLPKAQKLYLTHIDAEVEGDTHFPDYEPDDWESVFSEFHDADAQNSHSYCFEILERRGGSGGSGGMISLIAALAVDYVIGMENAMPWNLPADLAWFKRNTLNKPVIMGRHTWESIGRPLPGRKNIILSSQPSTDDRVTWVKSVDEAIAACGDVPEIMVIGGGRVIEQFLPKAQKLYLTHIDAEVEGDTHFPDYEPDDWESVFSEFHDADAQNSHSYCFEILERRGGSGGSGGMISLIAALAVDYVIGMENAMPWNLPADLAWFKRNTLNKPVIMGRHTWESIGRPLPGRKNIILSSQPSTDDRVTWVKSVDEAIAACGDVPEIMVIGGGRVIEQFLPKAQKLYLTHIDAEVEGDTHFPDYEPDDWESVFSEFHDADAQNSHSYCFEILERRGGSGGSGGMISLIAALAVDYVIGMENAMPWNLPADLAWFKRNTLNKPVIMGRHTWESIGRPLPGRKNIILSSQPSTDDRVTWVKSVDEAIAACGDVPEIMVIGGGRVIEQFLPKAQKLYLTHIDAEVEGDTHFPDYEPDDWESVFSEFHDADAQNSHSYCFEILERRGGSGGSGGMISLIAALAVDYVIGMENAMPWNLPADLAWFKRNTLNKPVIMGRHTWESIGRPLPGRKNIILSSQPSTDDRVTWVKSVDEAIAACGDVPEIMVIGGGRVIEQFLPKAQKLYLTHIDAEVEGDTHFPDYEPDDWESVFSEFHDADAQNSHSYCFEILERRGGSGGSGGMISLIAALAVDYVIGMENAMPWNLPADLAWFKRNTLNKPVIMGRHTWESIGRPLPGRKNIILSSQPSTDDRVTWVKSVDEAIAACGDVPEIMVIGGGRVIEQFLPKAQKLYLTHIDAEVEGDTHFPDYEPDDWESVFSEFHDADAQNSHSYCFEILERRGGSGGSGGMISLIAALAVDYVIGMENAMPWNLPADLAWFKRNTLNKPVIMGRHTWESIGRPLPGRKNIILSSQPSTDDRVTWVKSVDEAIAACGDVPEIMVIGGGRVIEQFLPKAQKLYLTHIDAEVEGDTHFPDYEPDDWESVFSEFHDADAQNSHSYCFEILERRGGSGGSGGMISLIAALAVDYVIGMENAMPWNLPADLAWFKRNTLNKPVIMGRHTWESIGRPLPGRKNIILSSQPSTDDRVTWVKSVDEAIAACGDVPEIMVIGGGRVIEQFLPKAQKLYLTHIDAEVEGDTHFPDYEPDDWESVFSEFHDADAQNSHSYCFEILERRGGSGGKLVPKKKRKVGGSNLLTVHQNLPALPVDATSDEVRKNLMDMFRDRQAFSEHTWKMLLSVCRSWAAWCKLNNRKWFPAEPEDVRDYLLYLQARGLAVKTIQQHLGQLNMLHRRSGLPRPSDSNAVSLVMRRIRKENVDAGERAKQALAFERTDFDQVRSLMENSDRCQDIRNLAFLGIAYNTLLRIAEIARIRVKDISRTDGGRMLIHIGRTKTLVSTAGVEKALSLGVTKLVERWISVSGVADDPNNYLFCRVRKNGVAAPSATSQLSTRALEGIFEATHRLIYGAKDDSGQRYLAWSGHSARVGAARDMARAGVSIPEIMQAGGWTNVNIVMNYIRNLDSETGAMVRLLEDGD

SEQ ID NO: 7 - protein, 2375 aa, Bacteriophage P1
  REGION 5..13: note = MISC_FEATURE - HAtag
  DOMAIN 21..179: note = Degradation Domain
  DOMAIN 188..346: note = Degradation Domain
  DOMAIN 355..513: note = Degradation Domain
  DOMAIN 522..680: note = Degradation Domain
  DOMAIN 689..847: note = Degradation Domain
  DOMAIN 856..1014: note = Degradation Domain
  DOMAIN 1023..1181: note = Degradation Domain
  DOMAIN 1190..1348: note = Degradation Domain
  DOMAIN 1357..1515: note = Degradation Domain
  DOMAIN 1524..1682: note = Degradation Domain
  DOMAIN 1691..1849: note = Degradation Domain
  DOMAIN 1858..2016: note = Degradation Domain
  SIGNAL 2024..2031: note = Nuclear Localization Signal
MVSKYPYDVPDYARSGGSGGMISLIAALAVDYVIGMENAMPWNLPADLAWFKRNTLNKPVIMGRHTWESIGRPLPGRKNIILSSQPSTDDRVTWVKSVDEAIAACGDVPEIMVIGGGRVIEQFLPKAQKLYLTHIDAEVEGDTHFPDYEPDDWESVFSEFHDADAQNSHSYCFEILERRGGSGGSGGMISLIAALAVDYVIGMENAMPWNLPADLAWFKRNTLNKPVIMGRHTWESIGRPLPGRKNIILSSQPSTDDRVTWVKSVDEAIAACGDVPEIMVIGGGRVIEQFLPKAQKLYLTHIDAEVEGDTHFPDYEPDDWESVFSEFHDADAQNSHSYCFEILERRGGSGGSGGMISLIAALAVDYVIGMENAMPWNLPADLAWFKRNTLNKPVIMGRHTWESIGRPLPGRKNIILSSQPSTDDRVTWVKSVDEAIAACGDVPEIMVIGGGRVIEQFLPKAQKLYLTHIDAEVEGDTHFPDYEPDDWESVFSEFHDADAQNSHSYCFEILERRGGSGGSGGMISLIAALAVDYVIGMENAMPWNLPADLAWFKRNTLNKPVIMGRHTWESIGRPLPGRKNIILSSQPSTDDRVTWVKSVDEAIAACGDVPEIMVIGGGRVIEQFLPKAQKLYLTHIDAEVEGDTHFPDYEPDDWESVFSEFHDADAQNSHSYCFEILERRGGSGGSGGMISLIAALAVDYVIGMENAMPWNLPADLAWFKRNTLNKPVIMGRHTWESIGRPLPGRKNIILSSQPSTDDRVTWVKSVDEAIAACGDVPEIMVIGGGRVIEQFLPKAQKLYLTHIDAEVEGDTHFPDYEPDDWESVFSEFHDADAQNSHSYCFEILERRGGSGGSGGMISLIAALAVDYVIGMENAMPWNLPADLAWFKRNTLNKPVIMGRHTWESIGRPLPGRKNIILSSQPSTDDRVTWVKSVDEAIAACGDVPEIMVIGGGRVIEQFLPKAQKLYLTHIDAEVEGDTHFPDYEPDDWESVFSEFHDADAQNSHSYCFEILERRGGSGGSGGMISLIAALAVDYVIGMENAMPWNLPADLAWFKRNTLNKPVIMGRHTWESIGRPLPGRKNIILSSQPSTDDRVTWVKSVDEAIAACGDVPEIMVIGGGRVIEQFLPKAQKLYLTHIDAEVEGDTHFPDYEPDDWESVFSEFHDADAQNSHSYCFEILERRGGSGGSGGMISLIAALAVDYVIGMENAMPWNLPADLAWFKRNTLNKPVIMGRHTWESIGRPLPGRKNIILSSQPSTDDRVTWVKSVDEAIAACGDVPEIMVIGGGRVIEQFLPKAQKLYLTHIDAEVEGDTHFPDYEPDDWESVFSEFHDADAQNSHSYCFEILERRGGSGGSGGMISLIAALAVDYVIGMENAMPWNLPADLAWFKRNTLNKPVIMGRHTWESIGRPLPGRKNIILSSQPSTDDRVTWVKSVDEAIAACGDVPEIMVIGGGRVIEQFLPKAQKLYLTHIDAEVEGDTHFPDYEPDDWESVFSEFHDADAQNSHSYCFEILERRGGSGGSGGMISLIAALAVDYVIGMENAMPWNLPADLAWFKRNTLNKPVIMGRHTWESIGRPLPGRKNIILSSQPSTDDRVTWVKSVDEAIAACGDVPEIMVIGGGRVIEQFLPKAQKLYLTHIDAEVEGDTHFPDYEPDDWESVFSEFHDADAQNSHSYCFEILERRGGSGGSGGMISLIAALAVDYVIGMENAMPWNLPADLAWFKRNTLNKPVIMGRHTWESIGRPLPGRKNIILSSQPSTDDRVTWVKSVDEAIAACGDVPEIMVIGGGRVIEQFLPKAQKLYLTHIDAEVEGDTHFPDYEPDDWESVFSEFHDADAQNSHSYCFEILERRGGSGGSGGMISLIAALAVDYVIGMENAMPWNLPADLAWFKRNTLNKPVIMGRHTWESIGRPLPGRKNIILSSQPSTDDRVTWVKSVDEAIAACGDVPEIMVIGGGRVIEQFLPKAQKLYLTHIDAEVEGDTHFPDYEPDDWESVFSEFHDADAQNSHSYCFEILERRGGSGGKLVPKKKRKVGGSNLLTVHQNLPALPVDATSDEVRKNLMDMFRDRQAFSEHTWKMLLSVCRSWAAWCKLNNRKWFPAEPEDVRDYLLYLQARGLAVKTIQQHLGQLNMLHRRSGLPRPSDSNAVSLVMRRIRKENVDAGERAKQALAFERTDFDQVRSLMENSDRCQDIRNLAFLGIAYNTLLRIAEIARIRVKDISRTDGGRMLIHIGRTKTLVSTAGVEKALSLGVTKLVERWISVSGVADDPNNYLFCRVRKNGVAAPSATSQLSTRALEGIFEATHRLIYGAKDDSGQRYLAWSGHSARVGAARDMARAGVSIPEIMQAGGWTNVNIVMNYIRNLDSETGAMVRLLEDGD

SEQ ID NO: 8 - protein, 400 aa, Bacteriophage P1
  REGION 5..13: note = MISC_FEATURE - HAtag
  SIGNAL 23..30: note = Nuclear Localization Signal
  REGION 33..183: note = MISC_FEATURE - N-terminal of Cre
  REGION 186..207: note = MISC_FEATURE - P2A
  REGION 210..400: note = MISC_FEATURE - C-terminal side of Cre
MVSKYPYDVPDYARSGGSGGKLVPKKKRKVGGSNLLTVHQNLPALPVDATSDEVRKNLMDMFRDRQAFSEHTWKMLLSVCRSWAAWCKLNNRKWFPAEPEDVRDYLLYLQARGLAVKTIQQHLGQLNMLHRRSGLPRPSDSNAVSLVMRRIRKENVDAGERAKQALAFERTDFDQVRSLMENSGSGSGATNFSLLKQAGDVEENPGPLEDRCQDIRNLAFLGIAYNTLLRIAEIARIRVKDISRTDGGRMLIHIGRTKTLVSTAGVEKALSLGVTKLVERWISVSGVADDPNNYLFCRVRKNGVAAPSATSQLSTRALEGIFEATHRLIYGAKDDSGQRYLAWSGHSARVGAARDMARAGVSIPEIMQAGGWTNVNIVMNYIRNLDSETGAMVRLLEDGD

SEQ ID NO: 9 - protein, 567 aa, Bacteriophage P1
  REGION 5..13: note = MISC_FEATURE - HAtag
  DOMAIN 21..179: note = Degradation Domain
  SIGNAL 190..197: note = Nuclear Localization Signal
  REGION 200..350: note = MISC_FEATURE - N-terminal side of Cre
  REGION 353..374: note = MISC_FEATURE - P2A
  REGION 377..567: note = MISC_FEATURE - C-terminal side of Cre
MVSKYPYDVPDYARSGGSGGMISLIAALAVDYVIGMENAMPWNLPADLAWFKRNTLNKPVIMGRHTWESIGRPLPGRKNIILSSQPSTDDRVTWVKSVDEAIAACGDVPEIMVIGGGRVIEQFLPKAQKLYLTHIDAEVEGDTHFPDYEPDDWESVFSEFHDADAQNSHSYCFEILERRGGSGGSGGKLVPKKKRKVGGSNLLTVHQNLPALPVDATSDEVRKNLMDMFRDRQAFSEHTWKMLLSVCRSWAAWCKLNNRKWFPAEPEDVRDYLLYLQARGLAVKTIQQHLGQLNMLHRRSGLPRPSDSNAVSLVMRRIRKENVDAGERAKQALAFERTDFDQVRSLMENSGSGSGATNFSLLKQAGDVEENPGPLEDRCQDIRNLAFLGIAYNTLLRIAEIARIRVKDISRTDGGRMLIHIGRTKTLVSTAGVEKALSLGVTKLVERWISVSGVADDPNNYLFCRVRKNGVAAPSATSQLSTRALEGIFEATHRLIYGAKDDSGQRYLAWSGHSARVGAARDMARAGVSIPEIMQAGGWTNVNIVMNYIRNLDSETGAMVRLLEDGD

SEQ ID NO: 10 - protein, 734 aa, Bacteriophage P1
  REGION 5..13: note = MISC_FEATURE - HAtag
  DOMAIN 21..179: note = Degradation Domain
  SIGNAL 190..197: note = Nuclear Localization Signal
  REGION 200..350: note = MISC_FEATURE - N-terminal side of Cre
  REGION 353..374: note = MISC_FEATURE - P2A
  REGION 377..567: note = MISC_FEATURE - C-terminal side of Cre
  DOMAIN 576..734: note = Degradation Domain
MVSKYPYDVPDYARSGGSGGMISLIAALAVDYVIGMENAMPWNLPADLAWFKRNTLNKPVIMGRHTWESIGRPLPGRKNIILSSQPSTDDRVTWVKSVDEAIAACGDVPEIMVIGGGRVIEQFLPKAQKLYLTHIDAEVEGDTHFPDYEPDDWESVFSEFHDADAQNSHSYCFEILERRGGSGGSGGKLVPKKKRKVGGSNLLTVHQNLPALPVDATSDEVRKNLMDMFRDRQAFSEHTWKMLLSVCRSWAAWCKLNNRKWFPAEPEDVRDYLLYLQARGLAVKTIQQHLGQLNMLHRRSGLPRPSDSNAVSLVMRRIRKENVDAGERAKQALAFERTDFDQVRSLMENSGSGSGATNFSLLKQAGDVEENPGPLEDRCQDIRNLAFLGIAYNTLLRIAEIARIRVKDISRTDGGRMLIHIGRTKTLVSTAGVEKALSLGVTKLVERWISVSGVADDPNNYLFCRVRKNGVAAPSATSQLSTRALEGIFEATHRLIYGAKDDSGQRYLAWSGHSARVGAARDMARAGVSIPEIMQAGGWTNVNIVMNYIRNLDSETGAMVRLLEDGDGGSGGSGGMISLIAALAVDYVIGMENAMPWNLPADLAWFKRNTLNKPVIMGRHTWESIGRPLPGRKNIILSSQPSTDDRVTWVKSVDEAIAACGDVPEIMVIGGGRVIEQFLPKAQKLYLTHIDAEVEGDTHFPDYEPDDWESVFSEFHDADAQNSHSYCFEILERR

SEQ ID NO: 11 - protein, 2401 aa, Bacteriophage P1
  REGION 5..13: note = MISC_FEATURE - HAtag
  DOMAIN 21..179: note = Degradation Domain
  DOMAIN 188..346: note = Degradation Domain
  DOMAIN 355..513: note = Degradation Domain
  DOMAIN 522..680: note = Degradation Domain
  DOMAIN 689..847: note = Degradation Domain
  DOMAIN 856..1014: note = Degradation Domain
  DOMAIN 1023..1181: note = Degradation Domain
  DOMAIN 1190..1348: note = Degradation Domain
  DOMAIN 1357..1515: note = Degradation Domain
  DOMAIN 1524..1682: note = Degradation Domain
  DOMAIN 1691..1849: note = Degradation Domain
  DOMAIN 1858..2016: note = Degradation Domain
  SIGNAL 2024..2031: note = Nuclear Localization Signal
  REGION 2034..2184: note = MISC_FEATURE - N-terminal side of Cre
  REGION 2187..2208: note = MISC_FEATURE - P2A
  REGION 2211..2401: note = MISC_FEATURE - C-terminal side of Cre
MVSKYPYDVPDYARSGGSGGMISLIAALAVDYVIGMENAMPWNLPADLAWFKRNTLNKPVIMGRHTWESIGRPLPGRKNIILSSQPSTDDRVTWVKSVDEAIAACGDVPEIMVIGGGRVIEQFLPKAQKLYLTHIDAEVEGDTHFPDYEPDDWESVFSEFHDADAQNSHSYCFEILERRGGSGGSGGMISLIAALAVDYVIGMENAMPWNLPADLAWFKRNTLNKPVIMGRHTWESIGRPLPGRKNIILSSQPSTDDRVTWVKSVDEAIAACGDVPEIMVIGGGRVIEQFLPKAQKLYLTHIDAEVEGDTHFPDYEPDDWESVFSEFHDADAQNSHSYCFEILERRGGSGGSGGMISLIAALAVDYVIGMENAMPWNLPADLAWFKRNTLNKPVIMGRHTWESIGRPLPGRKNIILSSQPSTDDRVTWVKSVDEAIAACGDVPEIMVIGGGRVIEQFLPKAQKLYLTHIDAEVEGDTHFPDYEPDDWESVFSEFHDADAQNSHSYCFEILERRGGSGGSGGMISLIAALAVDYVIGMENAMPWNLPADLAWFKRNTLNKPVIMGRHTWESIGRPLPGRKNIILSSQPSTDDRVTWVKSVDEAIAACGDVPEIMVIGGGRVIEQFLPKAQKLYLTHIDAEVEGDTHFPDYEPDDWESVFSEFHDADAQNSHSYCFEILERRGGSGGSGGMISLIAALAVDYVIGMENAMPWNLPADLAWFKRNTLNKPVIMGRHTWESIGRPLPGRKNIILSSQPSTDDRVTWVKSVDEAIAACGDVPEIMVIGGGRVIEQFLPKAQKLYLTHIDAEVEGDTHFPDYEPDDWESVFSEFHDADAQNSHSYCFEILERRGGSGGSGGMISLIAALAVDYVIGMENAMPWNLPADLAWFKRNTLNKPVIMGRHTWESIGRPLPGRKNIILSSQPSTDDRVTWVKSVDEAIAACGDVPEIMVIGGGRVIEQFLPKAQKLYLTHIDAEVEGDTHFPDYEPDDWESVFSEFHDADAQNSHSYCFEILERRGGSGGSGGMISLIAALAVDYVIGMENAMPWNLPADLAWFKRNTLNKPVIMGRHTWESIGRPLPGRKNIILSSQPSTDDRVTWVKSVDEAIAACGDVPEIMVIGGGRVIEQFLPKAQKLYLTHIDAEVEGDTHFPDYEPDDWESVFSEFHDADAQNSHSYCFEILERRGGSGGSGGMISLIAALAVDYVIGMENAMPWNLPADLAWFKRNTLNKPVIMGRHTWESIGRPLPGRKNIILSSQPSTDDRVTWVKSVDEAIAACGDVPEIMVIGGGRVIEQFLPKAQKLYLTHIDAEVEGDTHFPDYEPDDWESVFSEFHDADAQNSHSYCFEILERRGGSGGSGGMISLIAALAVDYVIGMENAMPWNLPADLAWFKRNTLNKPVIMGRHTWESIGRPLPGRKNIILSSQPSTDDRVTWVKSVDEAIAACGDVPEIMVIGGGRVIEQFLPKAQKLYLTHIDAEVEGDTHFPDYEPDDWESVFSEFHDADAQNSHSYCFEILERRGGSGGSGGMISLIAALAVDYVIGMENAMPWNLPADLAWFKRNTLNKPVIMGRHTWESIGRPLPGRKNIILSSQPSTDDRVTWVKSVDEAIAACGDVPEIMVIGGGRVIEQFLPKAQKLYLTHIDAEVEGDTHFPDYEPDDWESVFSEFHDADAQNSHSYCFEILERRGGSGGSGGMISLIAALAVDYVIGMENAMPWNLPADLAWFKRNTLNKPVIMGRHTWESIGRPLPGRKNIILSSQPSTDDRVTWVKSVDEAIAACGDVPEIMVIGGGRVIEQFLPKAQKLYLTHIDAEVEGDTHFPDYEPDDWESVFSEFHDADAQNSHSYCFEILERRGGSGGSGGMISLIAALAVDYVIGMENAMPWNLPADLAWFKRNTLNKPVIMGRHTWESIGRPLPGRKNIILSSQPSTDDRVTWVKSVDEAIAACGDVPEIMVIGGGRVIEQFLPKAQKLYLTHIDAEVEGDTHFPDYEPDDWESVFSEFHDADAQNSHSYCFEILERRGGSGGKLVPKKKRKVGGSNLLTVHQNLPALPVDATSDEVRKNLMDMFRDRQAFSEHTWKMLLSVCRSWAAWCKLNNRKWFPAEPEDVRDYLLYLQARGLAVKTIQQHLGQLNMLHRRSGLPRPSDSNAVSLVMRRIRKENVDAGERAKQALAFERTDFDQVRSLMENSGSGSGATNFSLLKQAGDVEENPGPLEDRCQDIRNLAFLGIAYNTLLRIAEIARIRVKDISRTDGGRMLIHIGRTKTLVSTAGVEKALSLGVTKLVERWISVSGVADDPNNYLFCRVRKNGVAAPSATSQLSTRALEGIFEATHRLIYGAKDDSGQRYLAWSGHSARVGAARDMARAGVSIPEIMQAGGWTNVNIVMNYIRNLDSETGAMVRLLEDGD

SEQ ID NO: 12 - DNA, 6953 bp, synthetic construct
  misc_feature 1..6953: note = Reporter gene expression cassette
  misc_feature 917..950: note = loxP sequence
  polyA_site 957..1752
  misc_feature 1759..1792: note = loxP sequence
  gene 1808..2512: note = mCherry
  source 1..6953: mol_type = other DNA
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttataacttcgtatagcatacattatacgaagttatggtaccgcagatctgcgactctagaggatctgcgactctagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgcgactctagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgcgactctagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatccccatcaagcatcgatataacttcgtatagcatacattatacgaagttatgaattcgccgccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaactcgaggggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 13 - DNA, 7877 bp, synthetic construct
  misc_feature 1..7877: note = Reporter gene expression cassette
  misc_feature 917..950: note = FRT site
  polyA_site 957..1752
  misc_feature 1759..1792: note = FRT site
  gene 1808..3436: note = firefly luciferase
  source 1..7877: mol_type = other DNA
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttgaagttcctatactttctagagaataggaacttcggtaccgcagatctgcgactctagaggatctgcgactctagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgcgactctagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgcgactctagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatccccatcaagcatcgatgaagttcctatactttctagagaataggaacttcgaattcgccgccatggaagatgccaaaaacattaagaagggcccagcgccattctacccactcgaagacgggaccgccggcgagcagctgcacaaagccatgaagcgctacgccctggtgcccggcaccatcgcctttaccgacgcacatatcgaggtggacattacctacgccgagtacttcgagatgagcgttcggctggcagaagctatgaagcgctatgggctgaatacaaaccatcggatcgtggtgtgcagcgagaatagcttgcagttcttcatgcccgtgttgggtgccctgttcatcggtgtggctgtggccccagctaacgacatctacaacgagcgcgagctgctgaacagcatgggcatcagccagcccaccgtcgtattcgtgagcaagaaagggctgcaaaagatcctcaacgtgcaaaagaagctaccgatcatacaaaagatcatcatcatggatagcaagaccgactaccagggcttccaaagcatgtacaccttcgtgacttcccatttgccacccggcttcaacgagtacgacttcgtgcccgagagcttcgaccgggacaaaaccatcgccctgatcatgaacagtagtggcagtaccggattgcccaagggcgtagccctaccgcaccgcaccgcttgtgtccgattcagtcatgcccgcgaccccatcttcggcaaccagatcatccccgacaccgctatcctcagcgtggtgccatttcaccacggcttcggcatgttcaccacgctgggctacttgatctgcggctttcgggtcgtgctcatgtaccgcttcgaggaggagctattcttgcgcagcttgcaagactataagattcaatctgccctgctggtgcccacactatttagcttcttcgctaagagcactctcatcgacaagtacgacctaagcaacttgcacgagatcgccagcggcggggcgccgctcagcaaggaggtaggtgaggccgtggccaaacgcttccacctaccaggcatccgccagggctacggcctgacagaaacaaccagcgccattctgatcacccccgaaggggacgacaagcctggcgcagtaggcaaggtggtgcccttcttcgaggctaaggtggtggacttggacaccggtaagacactgggtgtgaaccagcgcggcgagctgtgcgtccgtggccccatgatcatgagcggctacgttaacaaccccgaggctacaaacgctctcatcgacaaggacggctggctgcacagcggcgacatcgcctactgggacgaggacgagcacttcttcatcgtggaccggctgaagagcctgatcaaatacaagggctaccaggtagccccagccgaactggagagcatcctgctgcaacaccccaacatcttcgacgccggggtcgccggcctgcccgacgacgatgccggcgagctgcccgccgcagtcgtcgtgctggaacacggtaaaaccatgaccgagaaggagatcgtggactatgtggccagccaggttacaaccgccaagaagctgcgcggtggtgttgtgttcgtggacgaggtgcctaaaggactgaccggcaagttggacgcccgcaagatccgcgagattctcattaaggccaagaagtaactcgaggggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 14 - protein, 454 aa, Saccharomyces cerevisiae
  REGION 5..13: note = MISC_FEATURE - HAtag
  SIGNAL 23..30: note = Nuclear Localization Signal
  REGION 33..454: note = MISC_FEATURE - Flp
MVSKYPYDVPDYARSGGSGGKLVPKKKRKVGGSQFDILCKTPPKVLVRQFVERFERPSGEKIASCAAELTYLCWMITHNGTAIKRATFMSYNTIISNSLSFDIVNKSLQFKYKTQKATILEASLKKLIPAWEFTIIPYNGQKHQSDITDIVSSLQLQFESSEEADKGNSHSKKMLKALLSEGESIWEITEKILNSFEYTSRFTKTKTLYQFLFLATFINCGRFSDIKNVDPKSFKLVQNKYLGVIIQCLVTETKTSVSRHIYFFSARGRIDPLVYLDEFLRNSEPVLKRVNRTGNSSSNKQEYQLLKDNLVRSYNKALKKNAPYPIFAIKNGPKSHIGRHLMTSFLSMKGLTELTNVVGNWSDKRASAVARTTYTHQITAIPDHYFALVSRYYAYDPISKEMIALKDETNPIEEWQHIEQLKGSAEGSIRYPAWNGIISQEVLDYLSSYINRRI

SEQ ID NO: 15 - protein, 621 aa, Saccharomyces cerevisiae
  REGION 5..13: note = MISC_FEATURE - HAtag
  DOMAIN 21..179: note = Degradation Domain
  SIGNAL 190..197
  REGION 200..621: note = MISC_FEATURE - Flp
MVSKYPYDVPDYARSGGSGGMISLIAALAVDYVIGMENAMPWNLPADLAWFKRNTLNKPVIMGRHTWESIGRPLPGRKNIILSSQPSTDDRVTWVKSVDEAIAACGDVPEIMVIGGGRVIEQFLPKAQKLYLTHIDAEVEGDTHFPDYEPDDWESVFSEFHDADAQNSHSYCFEILERRGGSGGSGGKLVPKKKRKVGGSQFDILCKTPPKVLVRQFVERFERPSGEKIASCAAELTYLCWMITHNGTAIKRATFMSYNTIISNSLSFDIVNKSLQFKYKTQKATILEASLKKLIPAWEFTIIPYNGQKHQSDITDIVSSLQLQFESSEEADKGNSHSKKMLKALLSEGESIWEITEKILNSFEYTSRFTKTKTLYQFLFLATFINCGRFSDIKNVDPKSFKLVQNKYLGVIIQCLVTETKTSVSRHIYFFSARGRIDPLVYLDEFLRNSEPVLKRVNRTGNSSSNKQEYQLLKDNLVRSYNKALKKNAPYPIFAIKNGPKSHIGRHLMTSFLSMKGLTELTNVVGNWSDKRASAVARTTYTHQITAIPDHYFALVSRYYAYDPISKEMIALKDETNPIEEWQHIEQLKGSAEGSIRYPAWNGIISQEVLDYLSSYINRRI

SEQ ID NO: 16 - protein, 814 aa, Saccharomyces cerevisiae
  REGION 5..13: note = MISC_FEATURE - HAtag
  DOMAIN 21..179: note = Degradation Domain
  SIGNAL 190..197: note = Nuclear Localization Signal
  REGION 200..572: note = MISC_FEATURE - N-terminal side of Flp
  REGION 575..596: note = MISC_FEATURE - P2A
  REGION 599..647: note = MISC_FEATURE - C-terminal side of Flp
  DOMAIN 656..814: note = Degradation Domain
MVSKYPYDVPDYARSGGSGGMISLIAALAVDYVIGMENAMPWNLPADLAWFKRNTLNKPVIMGRHTWESIGRPLPGRKNIILSSQPSTDDRVTWVKSVDEAIAACGDVPEIMVIGGGRVIEQFLPKAQKLYLTHIDAEVEGDTHFPDYEPDDWESVFSEFHDADAQNSHSYCFEILERRGGSGGSGGKLVPKKKRKVGGSQFDILCKTPPKVLVRQFVERFERPSGEKIASCAAELTYLCWMITHNGTAIKRATFMSYNTIISNSLSFDIVNKSLQFKYKTQKATILEASLKKLIPAWEFTIIPYNGQKHQSDITDIVSSLQLQFESSEEADKGNSHSKKMLKALLSEGESIWEITEKILNSFEYTSRFTKTKTLYQFLFLATFINCGRFSDIKNVDPKSFKLVQNKYLGVIIQCLVTETKTSVSRHIYFFSARGRIDPLVYLDEFLRNSEPVLKRVNRTGNSSSNKQEYQLLKDNLVRSYNKALKKNAPYPIFAIKNGPKSHIGRHLMTSFLSMKGLTELTNVVGNWSDKRASAVARTTYTHQITAIPDHYFALVSRYYAYDPISKEMIALGSGSGATNFSLLKQAGDVEENPGPLEKDETNPIEEWQHIEQLKGSAEGSIRYPAWNGIISQEVLDYLSSYINRRIGGSGGSGGMISLIAALAVDYVIGMENAMPWNLPADLAWFKRNTLNKPVIMGRHTWESIGRPLPGRKNIILSSQPSTDDRVTWVKSVDEAIAACGDVPEIMVIGGGRVIEQFLPKAQKLYLTHIDAEVEGDTHFPDYEPDDWESVFSEFHDADAQNSHSYCFEILERR

SEQ ID NO: 17 - DNA, 7877 bp, synthetic construct
  misc_feature 1..7877: note = Reporter gene expression cassette
  misc_feature 917..950: note = Vlox site
  polyA_site 957..1752
  misc_feature 1759..1792: note = Vlox site
  gene 1808..3436: note = firefly luciferase
  source 1..7877: mol_type = other DNA
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagctttcaatttccgagaatgacagttctcagaaattgaggtaccgcagatctgcgactctagaggatctgcgactctagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgcgactctagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgcgactctagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatccccatcaagcatcgattcaatttccgagaatgacagttctcagaaattgagaattcgccgccatggaagatgccaaaaacattaagaagggcccagcgccattctacccactcgaagacgggaccgccggcgagcagctgcacaaagccatgaagcgctacgccctggtgcccggcaccatcgcctttaccgacgcacatatcgaggtggacattacctacgccgagtacttcgagatgagcgttcggctggcagaagctatgaagcgctatgggctgaatacaaaccatcggatcgtggtgtgcagcgagaatagcttgcagttcttcatgcccgtgttgggtgccctgttcatcggtgtggctgtggccccagctaacgacatctacaacgagcgcgagctgctgaacagcatgggcatcagccagcccaccgtcgtattcgtgagcaagaaagggctgcaaaagatcctcaacgtgcaaaagaagctaccgatcatacaaaagatcatcatcatggatagcaagaccgactaccagggcttccaaagcatgtacaccttcgtgacttcccatttgccacccggcttcaacgagtacgacttcgtgcccgagagcttcgaccgggacaaaaccatcgccctgatcatgaacagtagtggcagtaccggattgcccaagggcgtagccctaccgcaccgcaccgcttgtgtccgattcagtcatgcccgcgaccccatcttcggcaaccagatcatccccgacaccgctatcctcagcgtggtgccatttcaccacggcttcggcatgttcaccacgctgggctacttgatctgcggctttcgggtcgtgctcatgtaccgcttcgaggaggagctattcttgcgcagcttgcaagactataagattcaatctgccctgctggtgcccacactatttagcttcttcgctaagagcactctcatcgacaagtacgacctaagcaacttgcacgagatcgccagcggcggggcgccgctcagcaaggaggtaggtgaggccgtggccaaacgcttccacctaccaggcatccgccagggctacggcctgacagaaacaaccagcgccattctgatcacccccgaaggggacgacaagcctggcgcagtaggcaaggtggtgcccttcttcgaggctaaggtggtggacttggacaccggtaagacactgggtgtgaaccagcgcggcgagctgtgcgtccgtggccccatgatcatgagcggctacgttaacaaccccgaggctacaaacgctctcatcgacaaggacggctggctgcacagcggcgacatcgcctactgggacgaggacgagcacttcttcatcgtggaccggctgaagagcctgatcaaatacaagggctaccaggtagccccagccgaactggagagcatcctgctgcaacaccccaacatcttcgacgccggggtcgccggcctgcccgacgacgatgccggcgagctgcccgccgcagtcgtcgtgctggaacacggtaaaaccatgaccgagaaggagatcgtggactatgtggccagccaggttacaaccgccaagaagctgcgcggtggtgttgtgttcgtggacgaggtgcctaaaggactgaccggcaagttggacgcccgcaagatccgcgagattctcattaaggccaagaagtaactcgaggggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 18 - protein, 411 aa, Vibrio parahaemolyticus
  REGION 5..13: note = MISC_FEATURE - HAtag
  SIGNAL 23..30: note = Nuclear Localization Signal
  REGION 33..411: note = MISC_FEATURE - VCre
MVSKYPYDVPDYARSGGSGGKLVPKKKRKVGGIENQLSLLGDFSGVRPDDVKTAIQAAQKKGINVAENEQFKAAFEHLLNEFKKREERYSPNTLRRLESAWTCFVDWCLANHRHSLPATPDTVEAFFIERAEELHRNTLSVYRWAISRVHRVAGCPDPCLDIYVEDRLKAIARKKVREGEAVKQASPFNEQHLLKLTSLWYRSDKLLLRRNLALLAVAYESMLRASELANIRVSDMELAGDGTAILTIPITKTNHSGEPDTCILSQDVVSLLMDYTEAGKLDMSSDGFLFVGVSKHNTCIKPKKDKQTGEVLHKPITTKTVEGVFYSAWETLDLGRQGVKPFTAHSARVGAAQDLLKKGYNTLQIQQSGRWSSGAMVARYGRAILARDGAMAHSRVKTRSAPMQWGKDEKD

SEQ ID NO: 19 - protein, 578 aa, Vibrio parahaemolyticus
  REGION 5..13: note = MISC_FEATURE - HAtag
  DOMAIN 21..179: note = Degradation Domain
  SIGNAL 190..197: note = Nuclear Localization Signal
  REGION 200..578: note = MISC_FEATURE - VCre
MVSKYPYDVPDYARSGGSGGMISLIAALAVDYVIGMENAMPWNLPADLAWFKRNTLNKPVIMGRHTWESIGRPLPGRKNIILSSQPSTDDRVTWVKSVDEAIAACGDVPEIMVIGGGRVIEQFLPKAQKLYLTHIDAEVEGDTHFPDYEPDDWESVFSEFHDADAQNSHSYCFEILERRGGSGGSGGKLVPKKKRKVGGIENQLSLLGDFSGVRPDDVKTAIQAAQKKGINVAENEQFKAAFEHLLNEFKKREERYSPNTLRRLESAWTCFVDWCLANHRHSLPATPDTVEAFFIERAEELHRNTLSVYRWAISRVHRVAGCPDPCLDIYVEDRLKAIARKKVREGEAVKQASPFNEQHLLKLTSLWYRSDKLLLRRNLALLAVAYESMLRASELANIRVSDMELAGDGTAILTIPITKTNHSGEPDTCILSQDVVSLLMDYTEAGKLDMSSDGFLFVGVSKHNTCIKPKKDKQTGEVLHKPITTKTVEGVFYSAWETLDLGRQGVKPFTAHSARVGAAQDLLKKGYNTLQIQQSGRWSSGAMVARYGRAILARDGAMAHSRVKTRSAPMQWGKDEKD

SEQ ID NO: 20 - protein, 771 aa, Vibrio parahaemolyticus
  REGION 5..13: note = HAtag
  DOMAIN 21..179: note = Degradation Domain
  SIGNAL 190..197: note = Nuclear Localization Signal
  REGION 200..475: note = MISC_FEATURE - N-terminal side of VCre
  REGION 478..499: note = MISC_FEATURE - P2A
  REGION 502..604: note = MISC_FEATURE - C-terminal side of VCre
  DOMAIN 613..771: note = Degradation Domain
MVSKYPYDVPDYARSGGSGGMISLIAALAVDYVIGMENAMPWNLPADLAWFKRNTLNKPVIMGRHTWESIGRPLPGRKNIILSSQPSTDDRVTWVKSVDEAIAACGDVPEIMVIGGGRVIEQFLPKAQKLYLTHIDAEVEGDTHFPDYEPDDWESVFSEFHDADAQNSHSYCFEILERRGGSGGSGGKLVPKKKRKVGGIENQLSLLGDFSGVRPDDVKTAIQAAQKKGINVAENEQFKAAFEHLLNEFKKREERYSPNTLRRLESAWTCFVDWCLANHRHSLPATPDTVEAFFIERAEELHRNTLSVYRWAISRVHRVAGCPDPCLDIYVEDRLKAIARKKVREGEAVKQASPFNEQHLLKLTSLWYRSDKLLLRRNLALLAVAYESMLRASELANIRVSDMELAGDGTAILTIPITKTNHSGEPDTCILSQDVVSLLMDYTEAGKLDMSSDGFLFVGVSKHNTCIKPKKDKQTGSGSGATNFSLLKQAGDVEENPGPLEGEVLHKPITTKTVEGVFYSAWETLDLGRQGVKPFTAHSARVGAAQDLLKKGYNTLQIQQSGRWSSGAMVARYGRAILARDGAMAHSRVKTRSAPMQWGKDEKDGGSGGSGGMISLIAALAVDYVIGMENAMPWNLPADLAWFKRNTLNKPVIMGRHTWESIGRPLPGRKNIILSSQPSTDDRVTWVKSVDEAIAACGDVPEIMVIGGGRVIEQFLPKAQKLYLTHIDAEVEGDTHFPDYEPDDWESVFSEFHDADAQNSHSYCFEILERR

SEQ ID NO: 21 - DNA, 7873 bp, synthetic construct
  misc_feature 1..7873: note = Reporter gene expression cassette
  misc_feature 917..948: note = rox site
  gene 917..948: note = firefly luciferase
  polyA_site 955..1750
  misc_feature 1757..1788: note = rox site
  source 1..7873: mol_type = other DNA
cacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagctttaactttaaataattggcattatttaaagttaggtaccgcagatctgcgactctagaggatctgcgactctagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgcgactctagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgcgactctagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatccccatcaagcatcgattaactttaaataattggcattatttaaagttagaattcgccgccatggaagatgccaaaaacattaagaagggcccagcgccattctacccactcgaagacgggaccgccggcgagcagctgcacaaagccatgaagcgctacgccctggtgcccggcaccatcgcctttaccgacgcacatatcgaggtggacattacctacgccgagtacttcgagatgagcgttcggctggcagaagctatgaagcgctatgggctgaatacaaaccatcggatcgtggtgtgcagcgagaatagcttgcagttcttcatgcccgtgttgggtgccctgttcatcggtgtggctgtggccccagctaacgacatctacaacgagcgcgagctgctgaacagcatgggcatcagccagcccaccgtcgtattcgtgagcaagaaagggctgcaaaagatcctcaacgtgcaaaagaagctaccgatcatacaaaagatcatcatcatggatagcaagaccgactaccagggcttccaaagcatgtacaccttcgtgacttcccatttgccacccggcttcaacgagtacgacttcgtgcccgagagcttcgaccgggacaaaaccatcgccctgatcatgaacagtagtggcagtaccggattgcccaagggcgtagccctaccgcaccgcaccgcttgtgtccgattcagtcatgcccgcgaccccatcttcggcaaccagatcatccccgacaccgctatcctcagcgtggtgccatttcaccacggcttcggcatgttcaccacgctgggctacttgatctgcggctttcgggtcgtgctcatgtaccgcttcgaggaggagctattcttgcgcagcttgcaagactataagattcaatctgccctgctggtgcccacactatttagcttcttcgctaagagcactctcatcgacaagtacgacctaagcaacttgcacgagatcgccagcggcggggcgccgctcagcaaggaggtaggtgaggccgtggccaaacgcttccacctaccaggcatccgccagggctacggcctgacagaaacaaccagcgccattctgatcacccccgaaggggacgacaagcctggcgcagtaggcaaggtggtgcccttcttcgaggctaaggtggtggacttggacaccggtaagacactgggtgtgaaccagcgcggcgagctgtgcgtccgtggccccatgatcatgagcggctacgttaacaaccccgaggctacaaacgctctcatcgacaaggacggctggctgcacagcggcgacatcgcctactgggacgaggacgagcacttcttcatcgtggaccggctgaagagcctgatcaaatacaagggctaccaggtagccccagccgaactggagagcatcctgctgcaacaccccaacatcttcgacgccggggtcgccggcctgcccgacgacgatgccggcgagctgcccgccgcagtcgtcgtgctggaacacggtaaaaccatgaccgagaaggagatcgtggactatgtggccagccaggttacaaccgccaagaagctgcgcggtggtgttgtgttcgtggacgaggtgcctaaaggactgaccggcaagttggacgcccgcaagatccgcgagattctcattaaggccaagaagtaactcgaggggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 22 - protein, 373 aa, Bacteriophage D6
  REGION 5..13: note = MISC_FEATURE - HAtag
  SIGNAL 23..30: note = Nuclear Localization Signal
  REGION 33..373: note = MISC_FEATURE - Dre
MVSKYPYDVPDYARSGGSGGKLVPKKKRKVGGSELIISGSSGGFLRNIGKEYQEAAENFMRFMNDQGAYAPNTLRDLRLVFHSWARWCHARQLAWFPISPEMAREYFLQLHDADLASTTIDKHYAMLNMLLSHCGLPPLSDDKSVSLAMRRIRREAATEKGERTGQAIPLRWDDLKLLDVLLSRSERLVDLRNRAFLFVAYNTLMRMSEISRIRVGDLDQTGDTVTLHISHTKTITTAAGLDKVLSRRTTAVLNDWLDVSGLREHPDAVLFPPIHRSNKARITTTPLTAPAMEKIFSDAWVLLNKRDATPNKGRYRTWTGHSARVGAAIDMAEKQVSMVEIMQEGTWKKPETLMRYLRRGGVSVGANSRLMDS

SEQ ID NO: 23 - protein, 540 aa, Bacteriophage D6
  REGION 5..13: note = MISC_FEATURE - HAtag
  DOMAIN 21..179: note = Degradation Domain
  SIGNAL 190..197: note = Nuclear Localization Signal
  REGION 200..540: note = MISC_FEATURE - Dre
MVSKYPYDVPDYARSGGSGGMISLIAALAVDYVIGMENAMPWNLPADLAWFKRNTLNKPVIMGRHTWESIGRPLPGRKNIILSSQPSTDDRVTWVKSVDEAIAACGDVPEIMVIGGGRVIEQFLPKAQKLYLTHIDAEVEGDTHFPDYEPDDWESVFSEFHDADAQNSHSYCFEILERRGGSGGSGGKLVPKKKRKVGGSELIISGSSGGFLRNIGKEYQEAAENFMRFMNDQGAYAPNTLRDLRLVFHSWARWCHARQLAWFPISPEMAREYFLQLHDADLASTTIDKHYAMLNMLLSHCGLPPLSDDKSVSLAMRRIRREAATEKGERTGQAIPLRWDDLKLLDVLLSRSERLVDLRNRAFLFVAYNTLMRMSEISRIRVGDLDQTGDTVTLHISHTKTITTAAGLDKVLSRRTTAVLNDWLDVSGLREHPDAVLFPPIHRSNKARITTTPLTAPAMEKIFSDAWVLLNKRDATPNKGRYRTWTGHSARVGAAIDMAEKQVSMVEIMQEGTWKKPETLMRYLRRGGVSVGANSRLMDS

SEQ ID NO: 24 - protein, 733 aa, Bacteriophage D6
  REGION 5..13: note = MISC_FEATURE - HAtag
  DOMAIN 21..179: note = Degradation Domain
  SIGNAL 190..197: note = Nuclear Localization Signal
  REGION 200..349: note = MISC_FEATURE - N-terminal side of Dre
  REGION 352..373: note = MISC_FEATURE - P2A
  REGION 376..566: note = MISC_FEATURE - C-terminal side of Dre
  DOMAIN 575..733: note = Degradation Domain
MVSKYPYDVPDYARSGGSGGMISLIAALAVDYVIGMENAMPWNLPADLAWFKRNTLNKPVIMGRHTWESIGRPLPGRKNIILSSQPSTDDRVTWVKSVDEAIAACGDVPEIMVIGGGRVIEQFLPKAQKLYLTHIDAEVEGDTHFPDYEPDDWESVFSEFHDADAQNSHSYCFEILERRGGSGGSGGKLVPKKKRKVGGSELIISGSSGGFLRNIGKEYQEAAENFMRFMNDQGAYAPNTLRDLRLVFHSWARWCHARQLAWFPISPEMAREYFLQLHDADLASTTIDKHYAMLNMLLSHCGLPPLSDDKSVSLAMRRIRREAATEKGERTGQAIPLRWDDLKLLDVLLGSGSGATNFSLLKQAGDVEENPGPLESRSERLVDLRNRAFLFVAYNTLMRMSEISRIRVGDLDQTGDTVTLHISHTKTITTAAGLDKVLSRRTTAVLNDWLDVSGLREHPDAVLFPPIHRSNKARITTTPLTAPAMEKIFSDAWVLLNKRDATPNKGRYRTWTGHSARVGAAIDMAEKQVSMVEIMQEGTWKKPETLMRYLRRGGVSVGANSRLMDSGGSGGSGGMISLIAALAVDYVIGMENAMPWNLPADLAWFKRNTLNKPVIMGRHTWESIGRPLPGRKNIILSSQPSTDDRVTWVKSVDEAIAACGDVPEIMVIGGGRVIEQFLPKAQKLYLTHIDAEVEGDTHFPDYEPDDWESVFSEFHDADAQNSHSYCFEILERR

SEQ ID NO: 25 - DNA, 7075 bp, Synthetic construct
  source 1..7075: mol_type = other DNA
  misc_feature 1..7075: note = Reporter gene expression cassette
  gene 917..2545: note = In-frame stop codon-firefly luciferase
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagttaagcttgccaccatggaagatgccaaaaacattaagaagggcccagcgccattctacccactcgaagacgggaccgccggcgagcagctgcacaaagccatgaagcgctacgccctggtgcccggcaccatcgcctttaccgacgcacatatcgaggtggacattacctacgccgagtacttcgagatgagcgttcggctggcagaagctatgaagcgctatgggctgaatacaaaccatcggatcgtggtgtgcagcgagaatagcttgcagttcttcatgcccgtgttgggtgccctgttcatcggtgtggctgtggccccagctaacgacatctacaacgagcgcgagctgctgaacagcatgggcatcagccagcccaccgtcgtattcgtgagcaagaaagggctgcaaaagatcctcaacgtgcaaaagaagctaccgatcatacaaaagatcatcatcatggatagcaagaccgactaccagggcttccaaagcatgtacaccttcgtgacttcccatttgccacccggcttcaacgagtacgacttcgtgcccgagagcttcgaccgggacaaaaccatcgccctgatcatgaacagtagtggcagtaccggattgcccaagggcgtagccctaccgcaccgcaccgcttgtgtccgattcagtcatgcccgcgaccccatcttcggcaaccagatcatccccgacaccgctatcctcagcgtggtgccatttcaccacggcttcggcatgttcaccacgctgggctacttgatctgcggctttcgggtcgtgctcatgtaccgcttcgaggaggagctattcttgcgcagcttgcaagactataagattcaatctgccctgctggtgcccacactatttagcttcttcgctaagagcactctcatcgacaagtacgacctaagcaacttgcacgagatctaaagcggcggggcgccgctcagcaaggaggtaggtgaggccgtggccaaacgcttccacctaccaggcatccgccagggctacggcctgacagaaacaaccagcgccattctgatcacccccgaaggggacgacaagcctggcgcagtaggcaaggtggtgcccttcttcgaggctaaggtggtggacttggacaccggtaagacactgggtgtgaaccagcgcggcgagctgtgcgtccgtggccccatgatcatgagcggctacgttaacaaccccgaggctacaaacgctctcatcgacaaggacggctggctgcacagcggcgacatcgcctactgggacgaggacgagcacttcttcatcgtggaccggctgaagagcctgatcaaatacaagggctaccaggtagccccagccgaactggagagcatcctgctgcaacaccccaacatcttcgacgccggggtcgccggcctgcccgacgacgatgccggcgagctgcccgccgcagtcgtcgtgctggaacacggtaaaaccatgaccgagaaggagatcgtggactatgtggccagccaggttacaaccgccaagaagctgcgcggtggtgttgtgttcgtggacgaggtgcctaaaggactgaccggcaagttggacgcccgcaagatccgcgagattctcattaaggccaagaagtaactcgagtctagagggcccttcgaaggtaagcctatccctaaccctctcctcggtctcgattctacgcgtaccggtcatcatcaccatcaccattgagtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 26 - DNA, 5931 bp, synthetic construct
  source 1..5931: mol_type = other DNA
  misc_feature 1..5931: note = Reporter gene expression cassette
  gene 223..1851: note = Inverted firefly luciferase
tgtgctagcgcatatccagtgtagtaaggcaagtcccttcaagagttatcgttgatacccctcgtagtgcacattcctttaacgcttcaaaatctgtaaagcacgccatatcgccgaaaggcacacttaattattaagaggtaatacaccatgaatcacaaagtgcatcatcatcatcatcatatcgaaggtaggcatatggagctcggtaccctcgagttacttcttggccttaatgagaatctcgcggatcttgcgggcgtccaacttgccggtcagtcctttaggcacctcgtccacgaacacaacaccaccgcgcagcttcttggcggttgtaacctggctggccacatagtccacgatctccttctcggtcatggttttaccgtgttccagcacgacgactgcggcgggcagctcgccggcatcgtcgtcgggcaggccggcgaccccggcgtcgaagatgttggggtgttgcagcaggatgctctccagttcggctggggctacctggtagcccttgtatttgatcaggctcttcagccggtccacgatgaagaagtgctcgtcctcgtcccagtaggcgatgtcgccgctgtgcagccagccgtccttgtcgatgagagcgtttgtagcctcggggttgttaacgtagccgctcatgatcatggggccacggacgcacagctcgccgcgctggttcacacccagtgtcttaccggtgtccaagtccaccaccttagcctcgaagaagggcaccaccttgcctactgcgccaggcttgtcgtccccttcgggggtgatcagaatggcgctggttgtttctgtcaggccgtagccctggcggatgcctggtaggtggaagcgtttggccacggcctcacctacctccttgctgagcggcgccccgccgctggcgatctcgtgcaagttgcttaggtcgtacttgtcgatgagagtgctcttagcgaagaagctaaatagtgtgggcaccagcagggcagattgaatcttatagtcttgcaagctgcgcaagaatagctcctcctcgaagcggtacatgagcacgacccgaaagccgcagatcaagtagcccagcgtggtgaacatgccgaagccgtggtgaaatggcaccacgctgaggatagcggtgtcggggatgatctggttgccgaagatggggtcgcgggcatgactgaatcggacacaagcggtgcggtgcggtagggctacgcccttgggcaatccggtactgccactactgttcatgatcagggcgatggttttgtcccggtcgaagctctcgggcacgaagtcgtactcgttgaagccgggtggcaaatgggaagtcacgaaggtgtacatgctttggaagccctggtagtcggtcttgctatccatgatgatgatcttttgtatgatcggtagcttcttttgcacgttgaggatcttttgcagccctttcttgctcacgaatacgacggtgggctggctgatgcccatgctgttcagcagctcgcgctcgttgtagatgtcgttagctggggccacagccacaccgatgaacagggcacccaacacgggcatgaagaactgcaagctattctcgctgcacaccacgatccgatggtttgtattcagcccatagcgcttcatagcttctgccagccgaacgctcatctcgaagtactcggcgtaggtaatgtccacctcgatatgtgcgtcggtaaaggcgatggtgccgggcaccagggcgtagcgcttcatggctttgtgcagctgctcgccggcggtcccgtcttcgagtgggtagaatggcgctgggcccttcttaatgtttttggcatcttccatggtggcaagcttgtcgacctgcagtctagataggtaatctctgcttaaaagcacagaatctaagatccctgccatttggcggggatttttttatttgttttcaggaaataaataatcgatcgcgtaataaaatctattattatttttgtgaagaataaatttgggtgcaatgagaatgcgcaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataaaattgtaaacgttaatattttgttaaaattcgcgttaaatttttgttaaatcagctcattttttaaccaataggccgaaatcggcaaaatcccttataaatcaaaagaatagcccgagatagggttgagtgttgttccagtttggaacaagagtccactattaaagaacgtggactccaacgtcaaagggcgaaaaaccgtctatcagggcgatggcccactacgtgaaccatcacccaaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtactatggttgctttgacgtatgcggtgtgaaataccgcacagatgcgtaaggagaaaataccgcatcaggcgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgc

SEQ ID NO: 27 - protein, 1396 aa, Staphylococcus aureus
  SIGNAL 4..18: note = Nuclear Localization Signa
  SIGNAL 1388..1396: note = Nuclear Localization Signa
  REGION 19..1385: note = full-length spCas9
MGTPKKKRKVGIHGVPAADKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDEFASPKKKRKV

SEQ ID NO: 28 - protein, 1833 aa, Staphylococcus aureus
  SIGNAL 4..18: note = Nuclear Localization Signal
  REGION 19..730: note = Cas9N713
  MOTIF 738..766: note = Leucine zipper
  DOMAIN 777..936: note = Degradation Domain
  REGION 939..959: note = P2A
  DOMAIN 962..1120: note = Degradation Domain
  MOTIF 1131..1160: note = Leucine zipper
  REGION 1131..1160: note = Leucine zipper
  REGION 1168..1822: note = Cas9C714
  SIGNAL 1825..1833: note = Nuclear Localization Signal
MGTPKKKRKVGIHGVPAADKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVTGGGSGGALKKELQANKKELAQLKWELQALKKELAQGSGGSGGSGGMISLIAALAVDYVIGMENAMPWNLPADLAWFKRNTLNKPVIMGRHTWESIGRPLPGRKNIILSSQPSTDDRVTWVKSVDEAIAACGDVPEIMVIGGGRVIEQFLPKAQKLYLTHIDAEVEGDTHFPDYEPDDWESVFSEFHDADAQNSHSYCFEILERRRTGSGATNFSLLKQAGDVEENPGPIDMISLIAALAVDYVIGMENAMPWNLPADLAWFKRNTLNKPVIMGRHTWESIGRPLPGRKNIILSSQPSTDDRVTWVKSVDEAIAACGDVPEIMVIGGGRVIEQFLPKAQKLYLTHIDAEVEGDTHFPDYEPDDWESVFSEFHDADAQNSHSYCFEILERRGGSGGSGGLEEQLEKKLQALEKKLAQLEWKNQALEKKLAQGGSGGGTSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDEFASPKKKRKV

SEQ ID NO: 29 - DNA, 126 bp, synthetic construct
  source 1..126: mol_type = other DNA
  misc_feature 1..15: note = U6 promoter
  misc_feature 120..126: note = U6 terminator
tcttgtggaaaggacgaaacaccaacttgcacgagatctaaaggttttagagctagaaatagcaagttaaaataaggctagtccgttatcaacttgaaaaagtggcaccgagtcggtgcttttttt

SEQ ID NO: 30 - protein, 159 aa, Escherichia coli [sequence omitted]

SEQ ID NO: 31 - protein, 159 aa, Escherichia coli
MISLIAALAVDYVIGMENAMPWNLPADLAWFKRNTLNKPVIMGRHTWESIGRPLPGRKNIILSSQPSTDDRVTWVKSVDEAIAACGDVPEIMVIGGGRVIEQFLPKAQKLYLTHIDAEVEGDTHFPDYEPDDWESVFSEFHDADAQNSHSYCFEILERR

SEQ ID NO: 32 - protein, 108 aa, Homo sapiens
MGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLE

SEQ ID NO: 33 - protein, 108 aa, Homo sapiens
MGVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLE

SEQ ID NO: 34 - protein, 139 aa, unidentified
MVEKFVGTWKIADSHNFGEYLKAIGAPKELSDGGDATTPTLYISQKDGDKMTVKIENGPPTFLDTQVKFKLGEEFDEFPSDRRKGVKSVVNLVGEKLVYVQKWDGKETTYVREIKDGKLVVTLTMGDVVAVRSYRRATE

SEQ ID NO: 35 - protein, 187 aa, Mus musculus
MVRPLNCIVAVSQNMGIGKNGDLPWPPLRNEFKYFQRMTTTSSVEGKQNLVIMGRKTWFSIPEKNRPLKDRINIVLSRELKEPPRGAHFLAKSLDDALRLIEQPELASKVDMVWIVGGSSVYQEAMNQPGHLRLFVTRIMQEFESDTFFPEIDLGKYKLLPEYPGVLSEVQEEKGIKYKFEVYEKKD

SEQ ID NO: 36 - DNA, 34 bp, unidentified
  source 1..34: mol_type = other DNA
ataacttcgtatagcatacattatacgaagttat

SEQ ID NO: 37 - DNA, 34 bp, unidentified
  source 1..34: mol_type = other DNA
gaagttcctatactttctagagaataggaacttc

SEQ ID NO: 38 - DNA, 34 bp, unidentified
  source 1..34: mol_type = other DNA
tcaatttccgagaatgacagttctcagaaattga

SEQ ID NO: 39 - DNA, 32 bp, unidentified
  source 1..32: mol_type = other DNA
taactttaaataattggcattatttaaagtta

SEQ ID NO: 40 - DNA, 33 bp, unidentified
  source 1..33: mol_type = other DNA
aacttgcacgagatctaaagcggcggggcgccg